(19) United States
(12) Patent Application Publication
(10) Pub. No.: US 2012/0171286 A1
(43) Pub. Date: Jul. 5, 2012

(54) METHODS AND COMPOSITIONS FOR THE TREATMENT AND DIAGNOSIS OF STATIN-INDUCED MYOPATHY

(75) Inventors: Vikas P. Sukhatme, Newton, MA (US); Stewart H. Lecker, Brookline, MA (US); Jun-ichi Hanai, Boston, MA (US)

(73) Assignee: Beth Isreal Deaconess Medical Center, Boston, MA (US)

(21) Appl. No.: 13/266,355

(22) PCT Filed: Apr. 27, 2010
(86) PCT No.: PCT/US10/01229

§ 371 Date: Mar. 20, 2012

Related U.S. Application Data
(60) Provisional application No. 61/173,063, filed on Apr. 27, 2009.
(60) Provisional application No. 61/239,395, filed on Sep. 2, 2009.

(51) Int. Cl. A61K 9/20; A61K 31/19; A61K 31/66; A61K 31/366

(52) U.S. Cl. 424/464; 514/460; 514/557; 514/106

(57) ABSTRACT

Disclosed are methods for treating or preventing a statin-mediated myopathy in a subject via administration of a therapeutically effective amount of a geranylgeranylation activator. Further disclosed are kits containing a geranylgeranylation activator useful for the treatment of a statin-mediated myopathy.

### BACKGROUND OF THE INVENTION

The invention relates to methods and compositions for the treatment and diagnosis of statin-mediated myopathies.

Statins is the common name for the class of drugs termed 3-hydroxy-3-methylglutaryl coenzyme A (HMG CoA) reductase inhibitors. These drugs lower levels of low-density lipoprotein cholesterol and, as a treatment, have successfully reduced the risk of adverse cardiovascular events and coronary heart disease in dyslipidemic patient populations. Over 100 million patients worldwide are taking these drugs, and statin manufacturing and therapy is a multibillion-dollar industry. The success of statin therapy, however, has been tempered by drug-induced side effects including muscle toxicity. This condition has been termed statin-mediated myopathy and is a relatively infrequent, but often debilitating, complication of this otherwise successful therapeutic approach. The most serious of these complications is rhabdomyolysis, or muscle breakdown, which can result in kidney failure. More controversial is a milder version of this disease characterized by muscle pain (myalgias) or an inflammation of the muscles (myositis) with or without evidence of muscle damage as assessed by creatine kinase (CK) serum elevation. The incidence of statin-associated or -induced myalgia or myositis is generally thought to be between 1-5% of the patient population that is on statin therapy. However, some investigators believe that the incidence of these myopathies is much higher, between 10 and 20% of patients on statin therapeutic regimens.

The muscle atrophy program is well established in several disease states including advanced cancer (tumor cachexia), sepsis, diabetes, and other systemic diseases. Muscle atrophy is also a debilitating side effect associated with several additional disorders including inactivity, denervation, food deprivation, and fasting. More recently, a variety of drugs have been associated with varying degrees of myotoxicity, including myopathy, and ultimately muscle atrophy in its most severe form. The atrophy process usually manifests as a rapid loss of muscle mass where rates of cellular protein synthesis are surpassed by rates of cellular protein degradation. The cellular protein degradation program mobilizes muscle protein as a source of catabolic amino acids for gluconeogenesis or other stress-induced metabolic requirements. Despite diverging etiologies, myopathy and muscle atrophy in many diseases share common biochemical and transcriptional pathways, including ubiquitination and targeted proteosomal breakdown of muscle proteins.

Proteins destined for degradation by the ubiquitin (Ub)-proteasome pathway are first covalently linked to a chain of Ub molecules, which marks them for rapid breakdown to short peptides by the 26S proteasome. The key enzyme responsible for attaching Ub to protein substrates is a Ub-protein ligase (E3) that catalyzes the transfer of an activated form of Ub from a specific Ub-carrier protein (E2) to a lysine residue on the substrate protein. Individual E3s ubiquitinate specific classes of proteins; hence, the identity of the proteins degraded by the proteasome is largely determined by the complement of E3s active in individual cells. Atrogin-1 is one example of an E3 ligase that has been identified in muscle cells and shown to be upregulated in atrophying muscle cells. Increased expression of atrogin-1 has been implicated for a role in statin-induced myopathy and muscle atrophy.

There exists a need for therapeutics to prevent or treat statin-mediated myopathies.

### SUMMARY OF THE INVENTION

The invention provide a method of treating or reducing the likelihood (e.g., at least a 10%, 20%, 30%, 40%, 50%, 60%, 70%, 80%, 90%, 95%, or even a 100% reduction) of developing a statin-induced myopathy in a subject by administering a therapeutically effective amount of a geranylgeranylation activator in an amount and for a time sufficient to treat or reduce the likelihood of developing a statin-mediated myopathy in a subject (e.g., human). In different aspects of the invented method, administration of a geranylgeranylation activator results in at least a 10% reduction in one or more (e.g., 1, 2, 3, 4, 5, 6, 7, 8, 9, or 10) symptoms of a statin-mediated myopathy in a subject, results in at least a 10% reduction (e.g., at least a 20%, 30%, 40%, 50%, 60%, 70%, 80%, 90%, 95%, or even 100% reduction) in the likelihood of developing one or more symptoms of a statin-mediated myopathy in a subject, or prevents the development of one or more symptoms of a statin-mediated myopathy in a subject.

In an additional embodiment of the method of the invention, the geranylgeranylation activator is administered to a subject that has increased atrogin-1 expression (e.g., an at least 10%, 20%, 30%, 40%, 50%, 60%, 70%, 80%, 90%, 95%, or 100% increase, or at least a 2-fold, 3-fold, or 4-fold increase in atrogin-1 protein or mRNA expression) compared to the level of atrogin-1 expression in a subject that does not have a myopathy or is not receiving a statin.

In an additional embodiment of the method, the subject is administered a statin in addition to a geranylgeranylation activator. In different embodiments of the method, a geranylgeranylation activator is administered prior to administration of a statin, a geranylgeranylation activator is administered to the subject following administration of a statin or following cessation or termination of statin treatment, or a geranylgeranylation activator is administered simultaneously or sequentially with a statin.

In various embodiments of the methods of the invention, the geranylgeranylation activator is administered orally or parenterally (e.g., subcutaneous, intramuscular, or intravenous administration), administered daily or weekly, administered for extended release, and/or administered in a dose of between 10 μg/day to 500 mg day (e.g., 100 μg/day to 500 mg day, 1 mg/day to 400 mg/day, 25 mg/day to 300 mg/day, 25 mg/day to 250 mg/day, or 50 mg/day to 200 mg/day).

In any of the above methods of the invention, the statin may be administered orally or parenterally (e.g., subcutaneous, intramuscular, or intravenous administration), administered daily or weekly, administered for extended release, or administered in a dose of 1 mg/day to 400 mg/day (e.g., 1 mg/day to 350 mg/day, 10 mg/day to 350 mg/day, 50 mg/day to 300 mg/day, and 50 mg/day to 200 mg day).

The invention also provides kits containing a geranylgeranylation activator and instructions for administration of a geranylgeranylation activator for the treatment of a statin-induced myopathy in a subject. In different embodiments of the kit, instructions specify that the subject has increased atrogin-1 expression (e.g., an at least 10%, 20%, 30%, 40%, 50%, 60%, 70%, 80%, 90%, 95%, or 100% increase, or at least a 2-fold, 3-fold, or 4-fold increase in atrogin-1 protein or mRNA expression) compared to a subject that does not have a myopathy or is not receiving a statin.

In various embodiments of the kits of the invention, the geranylgeranylation activator may be formulated for oral or parenteral administration (e.g., subcutaneous, intramuscular, or intravenous administration), formulated for extended release, or administered in a dose of between 10 μg/day to 500 mg/day (e.g., 100 μg/day to 500 mg day, 1 mg/day to 400 mg/day, 25 mg/day to 300 mg/day, 25 mg/day to 250 mg/day, or 50 mg/day to 200 mg/day). In additional embodiments of the kits, the instructions may specify that the geranylgeranylation activator is administered daily or weekly.

The kits of the invention may further include a statin and instructions for administration of the statin for the treatment of a statin-induced myopathy. In various embodiments of the kits, the statin may be formulated for oral or parenteral administration (e.g., subcutaneous, intramuscular, or intravenous administration), formulated for extended release, formulated in the same dosage form with the geranylgeranylation activator, or provided in a dose of 1 mg/day to 400 mg/day (e.g., 1 mg/day to 350 mg/day, 10 mg/day to 350 mg/day, 50 mg/day to 300 mg/day, and 50 mg/day to 200 mg day).

In additional embodiments of the kits, the instructions may specify that a geranylgeranylation activator is administered prior to a statin, specify the co-administration of a geranylgeranylation activator and a statin, and/or specify that the statin be administered daily or weekly.

In different embodiments of all the above aspects of the invention, the geranylgeranylation activator is selected from the group of mevalonate, phosphomevalonate, 5-pyrophosphomevalonate, isopentenyl pyrophosphate, dimethylallyl pyrophosphate, farnesyl diphosphate, farnesyl diphosphate, geranyl diphosphate, geranylgeranyl pyrophosphate, and geranylgeranol.

In different examples of all the above embodiments of the invention, the one or more symptoms of a statin-induced myopathy include, but are not limited to, increased atrogin-1 expression or biological activity, increased creatine kinase (CK) enzyme levels, overt necrosis of myocytes, myalgia, myositis, myoskeletal pain, muscle pain, increased microglobinuria, or increased transaminase levels.

In different embodiments of all the above aspects of the invention, the statin may be, but is not limited to, simvastatin, atrovastatin, fluvastatin, pravastatin, rosuvastatin, pitavastatin, lovastatin, compactin, mevinolin, mevastatin, velostatin, synvinolin, rivastatin, and cerivastatin. In addition, in all the above aspects of the invention, the subject may be a mammal (e.g., a human).

In any of the above methods the subject may further be administered one or more atrogin-1 inhibitors (e.g., 1, 2, 3, 4, 5, 6, 7, 8, 9, or 10 atrogin-1 inhibitors). Any of the above kits may also include one or more atrogin-1 inhibitors (e.g., 1, 2, 3, 4, 5, 6, 7, 8, 9, or 10 atrogin-1 inhibitors). Non-limiting examples of atrogin-1 inhibitors in all the above aspects of the invention include a monoclonal or polyclonal anti-atrogin-1 antibody; an aptamer that specifically binds atrogin-1; an antisense nucleobase oligomer (e.g., 8 to 30 nucleotides in length) that contains a nucleic acid substantially identical to at least a portion of an atrogin-1 nucleic acid molecule, or a complementary sequence thereof; a morpholino oligomer that is complementary to at least a portion of an atrogin-1 nucleic acid molecule; or a small RNA (e.g., 15 to 32 nucleotides in length) having at least one strand that includes a nucleic acid sequence substantially identical to at least a portion of an atrogin-1 nucleic acid molecule (e.g., a sequence substantially identical to a translational start site or a splicing site of an atrogin-1 nucleic acid molecule), or a complementary sequence thereof.

By “alteration” is meant a change (i.e., increase or decrease). The alteration can indicate a change in the expression levels of an atrogin-1 nucleic acid or polypeptide as detected by standard art known methods such as those described below. As used herein, an alteration includes a 10% change in expression levels, preferably a 25% change, more preferably a 40%, 50%, 60%, 70%, 80%, 90%, 95%, 96%, 97%, 98%, 99%, or greater change in expression levels (e.g., at least a 2-fold, 3-fold, 4-fold, or 5-fold change). The alteration can also indicate a change (i.e., increase or decrease) in the biological activity of an atrogin-1 nucleic acid or polypeptide. As used herein, an alteration includes a 10% change in biological activity, preferably a 25% change, more preferably a 40%, 50%, 60%, 70%, 80%, 90%, 95%, 96%, 97%, 98%, 99%, or greater change in biological activity (e.g., at least a 2-fold, 3-fold, 4-fold, or 5-fold change). Examples of biological activity for atrogin-1 polypeptides are described below. Alteration of atrogin-1 activity or expression levels in a cell may be compared to a control cell not treated with a statin.

By “atrogene” or “atrogenes” is meant a member of the family of proteins or a nucleic acid encoding the proteins, involved in the common biochemical and transcriptional atrophy program. This includes, but is not limited to, atrogin-1, a F-box protein regulated by the Forkhead box O (Foxo) family of transcription factors, which are also may be acknowledged as atrogenes. Foxo-1 induction has been demonstrated in most, if not all forms of atrophy. Foxo-3 (FoxO3) has been shown to regulate the expression of atrogin-1, and as such, is also included in the family of atrogenes. The Foxo family of proteins are tightly regulated by PI3K/AKT dependent phosphorylation, which may be considered upstream accessory components of the atrophy program. An additional member of the atrogene family may be MuRF-1, an additional ubiquitin E3 ligase.

By “atrogin-1” is meant a polypeptide, or a nucleic acid sequence that encodes it, or fragments or derivatives thereof, that is substantially identical to atrogin-1 nucleic acid or polypeptide sequences set forth in Genbank Accession Nos: BC024030 and AAH24030 (human atrogin-1 mRNA (FIG. 1) and protein (FIG. 2), respectively), BC027211 and AAH27211 (mouse atrogin-1 mRNA and protein, respectively), or zebrafish atrogin-1 protein (sequence published in International Patent Application No. PCT/US08/007047). A diagram of the atrogin-1 polypeptide is depicted in FIG. 3. Atrogin-1 can also include fragments, derivatives, homologs, sequence variants, splice variants, or analogs of atrogin-1 that retain at least 25%, 30%, 40%, 50%, 60%, 70%, 80%, 90%, 95%, 96%, 97%, 98%, 99%, or more atrogin-1 biological activity.

An atrogin-1 polypeptide or nucleic acid molecule may be isolated from a variety of sources, such as from mammalian tissue or cells (e.g., myocytes) or from another source, or prepared by recombinant or synthetic methods. The term “atrogin-1” also encompasses modifications to the polypeptide, fragments, derivatives, analogs, and variants of the atrogin-1 polypeptide.

“Atrogin-1 biological activity” can include one or more of the following exemplary activities: substrate binding activity (e.g., calcineurin A), ubiquitin ligase activity, inhibition of calcineurin A activity, and nuclear translocation. Assays for atrogin-1 biological activity include assays for ubiquitination, substrate binding assays, calcineurin A activity assays, nuclear translocation, and other assays (e.g., assays described in International Patent Application No. PCT/US08/007047, herein incorporated by reference).

By “atrogin-1 inhibitor compound” is meant any small molecule chemical compound (peptidyl or non-peptidyl), antibody, nucleic acid molecule, polypeptide, or fragments thereof that reduces or inhibits the expression levels or biological activity of atrogin-1 by at least 10%, 20%, 30%, 40%, 50%, 60%, 70%, 80%, 90%, or more. Non-limiting examples of atrogin-1 inhibitor compounds include fragments of atrogin-1 (e.g., dominant negative fragments or fragments that lack or have decreased ubiquitin ligase activity, substrate binding activity, calcineurin A inhibition, or nuclear translocation); peptidyl or non-peptidyl compounds that specifically bind atrogin-1 (e.g., antibodies or antigen-binding fragments thereof), for example, at the ubiquitin ligase domain, substrate binding domain, or the N- or C-terminal nuclear localization sequences of atrogin-1 (amino acids 62-66 and amino acids 267-288 of atrogin-1); antisense nucleobase oligomers; morpholino oligonucleotides or those molecules which target the translation start sequence or splicing sequence of an atrogin-1 mRNA); small RNAs; small molecule inhibitors; compounds that decrease the half-life of atrogin-1 mRNA or protein; compounds that decrease transcription or translation of atrogin-1; compounds that reduce or inhibit the expression levels of atrogin-1 polypeptides or decrease the biological activity of atrogin-1 polypeptides (e.g., PGC-1α or PGC-1β protein); compounds that increase the expression or biological activity of a second atrogin-1 inhibitor (e.g., a molecule which increases the transcription or translation of PGC-1α or PGC-1β protein such as metformin); compounds that block atrogin-1 -mediated downstream activities (e.g., ubiquitination; binding to SCF family members Skp-1 Cul-1, Roc-1; inhibition of calcineurin A activity; and nuclear translocation), and any compound that alters activities upstream of atrogin-1 (e.g., Foxo phosphorylation and PI3K/Akt phophorylation). Desirable atrogin-1 inhibitor compounds mediate at least a 10%, 15%, 20%, 25%, 30%, 35%, 40%, 45%, 50%, 55%, 60%, 65%, 70%, 75%, 80%, 85%, 90%, 95% or more decrease in atrogin-1 expression level or activity as compared to a control where the compound has not been added. Examples of atrogin-1 inhibitor compounds are described in International Patent Application No. PCT/US08/007047 (herein incorporated in its entirety). The geranyl-geranylation activators described herein are examples of atrogin-1 inhibitor compounds.

By “decrease” is meant to reduce, preferably by at least 20%, more preferably by at least 30%, and most preferably by at least 40%, 50%, 60%, 70%, 75%, 80%, 85%, 90%, 95%, or more. Decrease can refer, for example, to one or more symptoms of a statin-induced myopathy. Decrease may also refer to a decrease in the induction of atrogin-1 expression and/or biological activity (e.g., a decrease in the expression and/or biological activity of atrogin-1 in a cell treated with a statin and a geranylgeranylation activator compared to the expression and/or activity of atrogin-1 in a cell treated with a statin alone).

By “effective amount” is meant an amount sufficient to prevent or reduce the likelihood of developing a statin-mediated myopathy or any symptom associated with a statin-mediated myopathy. It will be appreciated that there will be many ways known in the art to determine the therapeutic amount for a given application. For example, the pharmacological methods for dosage determination may be used in the therapeutic context.

By “efficacy” is meant the effectiveness of a particular treatment regime. For example, efficacy in treating or reducing the likelihood of developing a statin-mediated myopathy can be measured by a reduction in any one or more of the following clinical or subclinical symptoms: atrogin-1 expression and/or biological activity, creatine kinase (CK) enzyme levels, overt necrosis of myocytes as evidenced by muscle biopsy, myalgia, musculoskeletal pain, muscle pain, musculoskeletal/connective tissue symptoms, or reduction of microglobinuria or transaminase levels with respect to rhabdomyolysis and hepatotoxicity.

By “expression” is meant the detection of a nucleic acid molecule or polypeptide by standard art known methods. For example, polypeptide expression is often detected by Western blotting, DNA expression is often detected by Southern blotting or polymerase chain reaction (PCR), and RNA expression is often detected by Northern blotting, RT-PCR, or RNAse protection assays.

By “extended release” is meant formulation of a statin compound or geranylgeranylation activator such that the release of the active agent (e.g., statin compound or geranylgeranylation activator), when in combination with another non-active substance (e.g., binder, filler, protein, or polymer), into a physiological buffer (e.g., water or phosphate buffered saline) is decreased relative to the agent's rate of diffusion through a physiological buffer when the agent is not formulated with a non-active substance. Extended release formulations may decrease the rate of release of a statin compound and/or geranylationgeranylation activator by at least 10%, 20%, 30%, 40%, 50%, 60%, 70%, 80%, 90%, or 100% compared to the rate of release of a statin and/or geranylgeranylation activator formulation which does not contain a non-active substance (e.g., binder, filler, protein, or polymer).

By “geranylgeranylation activator” is meant any small molecule, metabolite, protein, or nucleic acid sequence (e.g., DNA or RNA) that increases protein geranylgeranylation, increases the activity of geranylgeranylated proteins, or increases the activity of one or more enzymes in geranylgeranylation biosynthetic pathways in the cell. For example, a geranylgeranylation activator may increase the geranylgeranylation and/or activity of one or more (e.g., 1, 2, 3, or 4) signaling proteins including, but not limited to, small GTPases (e.g., rac, rho, rheb, and rap1). A geranylgeranylation activator may also mediate an increase in the activity or expression level of one or more (e.g., 1, 2, 3, 4, 5, 6, 7, 8, or 9) enzymes in geranylgeranylation biosynthetic pathways, e.g., mevalonate kinase, phosphomevalonate kinase, diphosphomevalonate decarboxylase, isopentenyl-diphosphate delta-isomerase, farnesyl diphosphate synthase (also known as farnesyl pyrophosphate synthetase, dimethylallyltranstransferase, and geranyltranstransferase), geranylgeranyl diphosphate synthase 1, farnesyltransferase, protein geranylgeranyltransferase, rab geranylgeranyltransferase, and isoprenylcysteine carboxyl methyltransferase (e.g., a combination of one or more small molecules, metabolites, signaling molecules, or hormones that activate the expression and/or activity of one or more enzymes in a geranylgeranylation biosynthetic pathway). An example of two geranylgeranylation activators that increase the expression of an enzyme in a geranylgeranylation biosynthetic pathway is the combination of insulin and thyropropin, which mediates an increase in the expression of geranylgeranyl pyrophosphate synthase (Fuse et al., Biochem. Biophys. Res. Comm. 315:1147-1153, 2004).

A geranylgeranylation activator may be a nucleic acid, e.g., a nucleic acid that contains a sequence encoding a polypeptide at least 85% identical (e.g., at least 90%, 95%, 99%, or even 100% identical) to one or more enzymes in a geranylgeranylation biosynethic pathway, such as mevalonate kinase, phosphomevalonate kinase, diphosphomevalonate decarboxylase, isopentenyl-diphosphate delta-isomerase, dimethyltranstransferase, farnesyl diphosphate synthase, geranylgeranyl diphosphate synthase 1, farnesyltransferase, protein geranylgeranyltransferase, rab geranylgeranyltransferase, and isoprenylcysteine carboxyl methyltransferase, or an activate fragment thereof A geranylgeranylation activator may also be a purified protein containing a sequence that is at least 85% identical (e.g., 90%, 95%, 99%, or even 100% identical) to an enzyme in a geranylgeranylation biosynethetic pathway (e.g., mevalonate kinase, phosphomevalonate kinase, diphosphomevalonate decarboxylase, isopentenyl-diphosphate delta-isomerase, dimethyltranstransferase, farnesyl diphosphate synthase, geranylgeranyl diphosphate synthase 1, farnesyltransferase, protein geranylgeranyltransferase, rab geranylgeranyltransferase, and isoprenylcysteine carboxyl methyltransferase), or an active fragment thereof.

A geranylgeranylation activator may also be a small molecule or metabolite in the geranylgeranylation biosynthetic pathway, including but not limited to, mevalonate, phosphomevalonate, 5-pyrophosphomevalonate, isopentenyl pyrophosphate, dimethylallyl pyrophosphate, farnesyl diphosphate, geranyl diphosphate, geranylgeranyl pyrophosphate, or geranylgeranol, or compounds which will breakdown to yield these molecules (e.g., a salt comprising one of these molecules).

Additional geranylgeranylation activators include one or biological agents (e.g., small molecules, metabolites, polypeptides, signaling proteins, or hormones) that increase the expression and/or activity of one or more enzymes (e.g., 1, 2, 3, 4, 5, 6, 7, 8, 9, or 10) in a geranylgeranylation biosynthetic pathway (e.g., mevalonate kinase, phosphomevalonate kinase, diphosphomevalonate decarboxylase, isopentenyl-diphosphate delta-isomerase, dimethyltranstransferase, farnesyl diphosphate synthase, geranylgeranyl diphosphate synthase 1, farnesyltransferase, protein geranylgeranyltransferase, rab geranylgeranyltransferase, and isoprenylcysteine carboxyl methyltransferase).

By “high dosage” of a statin compound is meant administration of a statin compound to a subject at a total dose of greater than 10 mg/day, 20 mg/day, 30 mg/day, 40 mg/day, 50 mg/day, 60 mg/day, 70 mg/day, or 80 mg/day. The total dose administered to a patient in a single day may occur via separate dose units (examples include, but are not limited to, two pills, three pills, and a cream and a pill). A patient may be treated with a high dosage of a statin compound for any duration of time (e.g., more than one day, more than one week, more than one month, and more than one year). Any of the statins described herein are contemplated for administration at high dosage.

By “increase” is meant to augment, preferably by at least 20%, more preferably by at least 50%, and most preferably by at least 70%, 75%, 80%, 85%, 90%, 95%, or more. Increase can refer, for example, to the levels and/or biological activity of atrogin-1.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of atrogin-1 polypeptide in a subject having a statin-mediated myopathy and a normal reference subject. Depending on the metric that is used, the diagnostic indicator of a statin-mediated myopathy may be significantly above or below a reference value (e.g., from a control subject not having myopathy or undergoing statin therapy).

By “myopathy” is meant a disease of the muscle or muscle tissue. Non-limiting examples include congenital myopathies, muscular dystrophies, inflammatory myopathies, mitochondrial myopathies (e.g., Kearns-Sayre syndrome, MELAS, and MERRF), Pompe's disease, Andersen's disease, Cori's disease, myoglobinureas (e.g., McArdle, Tarui, and DiMauro diseases), myositis ossificans, dermatomyositis, familial periodic paralysis, polymyositis, inclusion body myositis, neuromyotonia, stiff-man syndrome, and tetany. The methods of the invention may be used to treat or reduce the likelihood of developing a myopathy (e.g., a statin-induced myopathy).

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), Ed. A. Gennaro, 2000, Lippincott, Williams & Wilkins, Philadelphia, Pa.

By “positive reference” is meant a biological sample, for example, a biological fluid (e.g., urine, blood, serum, plasma, or cerebrospinal fluid), tissue (e.g., muscle tissue), or cell (e.g., myocyte), collected from a subject who has a myopathy (e.g., a statin-induced myopathy) or a propensity to develop a myopathy (e.g., a statin-induced myopathy). A positive reference may also be a biological sample derived from a patient with a statin-induced myopathy prior to or during treatment. In addition, a positive reference may be derived from a subject that is known to have a statin-mediated myopathy, that is matched to the sample subject by at least one of the following criteria: age, weight, BMI, disease stage, overall health, prior diagnosis of a statin-mediated myopathy, and a family history of statin-mediated myopathy. A positive reference as used herein may also be purified atrogin-1 polypeptide (e.g., recombinant or non-recombinant atrogin-1 polypeptide), purified atrogin-1 nucleic acid, purified anti-atrogin-1 antibody, or any biological sample (e.g., a biological fluid, tissue, or cell) that contains atrogin-1 polypeptide, atrogin-1 nucleic acid, or an anti-atrogin-1 antibody. A standard curve of levels of purified atrogin-1 protein, purified atrogin-1 nucleic acid, or an anti-atrogin-1 antibody within a positive reference range can also be used as a reference.

By “preventing” is meant prophylactic treatment of a subject who is not yet ill, but who is susceptible to, or otherwise at risk of, developing a particular disease. Preferably, a subject is determined to be at risk of developing a statin-induced myopathy. “Preventing” can refer to the preclusion of a statin-mediated myopathy in a patient receiving a statin compound for the treatment or prevention of elevated blood cholesterol levels (e.g., LDL), hyperlipidemia, heart disease, stroke, heart attack, atherosclerosis, intermittent claudication, hypertension, coronary artery disease, type 1 (insulin dependent diabetes or IDDM) and type 2 (non-insulin-dependent diabetes or NIDDM) diabetes, and other related disease states. For example, the preventive measures are used to prevent a statin-mediated myopathy in a patient undergoing a statin therapy with a statin that has been identified as, or associated with, developing myopathy in the patient population. “Preventing” can also refer to the preclusion of the worsening of the symptoms of a statin-induced myopathy. For example, a compound of the invention can be used to prevent a mild statin myopathy (e.g., characterized by muscle pain (myalgias) or an inflammation of the muscles (myositis)) from developing into rhabdomyolysis (i.e. or e.g., muscle breakdown).

By “protein,” “polypeptide,” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “reduce or inhibit” is meant the ability to cause an overall decrease preferably of 20% or greater, more preferably of 50% or greater, and most preferably of 65%, 70%, 75%, 80%, 85%, 90%, 95%, or greater. Reduce or inhibit can refer to the symptoms of the statin-mediated myopathy being treated, the levels of atrogin-1 polypeptide or nucleic acid, the levels of creatine kinase measured in a patient sample, or the amount of muscle or tissue loss in advanced or more serious statin-mediated myopathies (rhabdomyolysis). For diagnostic or monitoring applications, reduce or inhibit can refer to the level of protein or nucleic acid, detected by the aforementioned assays (see “expression”).

By “reducing the likelihood” is meant a reduction (e.g., at least 5%, 10%, 15%, 20%, 25%, 30%, 35%, 40%, 50%, 60%, 70%, 80%, 90%, or even 100% reduction) in the chance of developing a statin-induced myopathy or a reduction in the chance of development of one or more symptoms (e.g., 2, 3, 4, 5, 6, 7, 8, 9, or 10) of a statin-induced myopathy (e.g., increased atrogin-1 expression or biological activity, increased creatine kinase (CK) enzyme levels, overt necrosis of myocytes, myalgia, myositis, myoskeletal pain, muscle pain, or increased microglobinuria or transaminase levels). The likelihood of developing a statin-induced myopathy or the likelihood of developing one or more symptoms of a statin-induced myopathy in a subject treated with a geranylgeranylation activator may be compared to the likelihood of developing a statin-induced myopathy or the likelihood of developing one or more symptoms of a statin-induced myopathy in a subject (or a population of subjects) not treated with a geranylgeranylation activator, but receiving a statin.

By “reference sample” is meant any sample, standard, or level that is used for comparison purposes. A “normal reference sample” can be, for example, a prior sample taken from the same subject; a sample from a subject not having a statin-mediated myopathy; a subject not treated with a statin; a subject that is diagnosed with a propensity to develop a statin-mediated myopathy but does not yet show symptoms of the disorder; a subject that has been treated for a statin-mediated myopathy; or a sample of a purified reference atrogin-1 polypeptide or nucleic acid molecule at a known normal concentration. By “reference standard or level” is meant a value or number derived from a reference sample. A normal reference standard or level can be a value or number derived from a normal subject who does not have a statin-mediated myopathy. In preferred embodiments, the reference sample, standard, or level is matched to the sample subject by at least one of the following criteria: age, weight, body mass index (BMI), disease stage, and overall health. A “positive reference” sample, standard, or value is a sample, value, or number derived from a subject that is known to have a statin-mediated myopathy, that is matched to the sample subject by at least one of the following criteria: age, weight, BMI, disease stage, overall health, prior diagnosis of a statin-mediated myopathy, and a family history of statin-mediated myopathy. A standard curve of levels of purified protein within the normal or positive reference range can also be used as a reference.

By “sample” is meant a bodily fluid (e.g., urine, blood, serum, plasma, or cerebrospinal fluid), tissue (e.g., muscle tissue), or cell (e.g., myocyte) in which the atrogin-1 polypeptide or nucleic acid molecule is normally detectable.

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds atrogin-1 does not bind other ubiquitin ligase or atrogene family members.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., a atrogin-1 sequence or an enzyme of a geranylgeranylation biosynthetic pathway, such as those described herein and known in the art. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman, J. Mol. Biol. 147:195-7, 1981); “BestFit” (Smith and Waterman, Advances in Applied Mathematics, 482-489, 1981) as incorporated into GeneMatcher Plus™, Schwarz and Dayhof, “Atlas of Protein Sequence and Structure,” Dayhof, M. O., Ed pp 353-358, 1979; BLAST program (Basic Local Alignment Search Tool; (Altschul, S. F., W. Gish, et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 160, 170, 180, 190, 200, 209 amino acids or more. For nucleic acids, the length of comparison sequences will generally be at least 10, 20, 30, 40, 50, 60, 70, 80, 90, or 100, 200, 300, 400, 500, 600, 627, or more nucleotides. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “treating” is meant administering a compound or a pharmaceutical composition for prophylactic and/or therapeutic purposes or administering treatment to a subject already suffering from a disease to improve the subject's condition or to a subject who is at risk of developing a disease. As it pertains to statin-mediated myopathies, treating can include improving or ameliorating the symptoms of a statin-mediated myopathy and prophylactic treatment can include preventing the progression of a mild myopathy (e.g., myalgia and myositis) to a more serious form such as rhabdomyolysis. Prophylactic treatment can be monitored, for e.g., by measuring the CK levels in a subject undergoing prophylactic treatment and ensuring that the CK levels do not become significantly elevated or, desirably, to cause a detectable decrease in the CK levels. Treating may also mean to prevent the onset of a myopathy or the symptoms of a myopathy in a patient receiving a statin or a patient identified as at risk for developing a statin-induced myopathy (e.g., using the methods described herein).

By “statin-mediated myopathy” is meant the presence of clinical or subclinical symptoms of myopathy in a patient undergoing statin therapy. This includes muscle pain (myalgia) or an inflammation of the muscles (myositis) with or without evidence of muscle damage as assessed by CK elevations in the blood, serum, or plasma. This may accompany histological confirmation of necrosis by muscle biopsy. The recent American College of Cardiology/American Heart Association clinical advisory on the use and safety of statins defined four syndromes: statin myopathy (any muscle complaints related to these drugs); myalgia (muscle complaints without serum CK elevations); myositis (muscle symptoms with CK elevations); and rhabdomyolysis (markedly elevated CK levels, usually >10 times the upper limit of normal (ULN), with an elevated creatinine level consistent with pigment-induced nephropathy).

By a “statin compound” or “statin” is meant any a pharmaceutical compound that inhibits HMG-CoA reductase. Generally, statin compounds are understood to be those active agents which may be used to lower the lipid levels, including cholesterol, in the blood of a subject. The class of HMG-CoA reductase inhibitors includes both naturally occurring and synthetic molecules having differing structural features. Exemplary HMG-CoA reductase inhibitors include: atorvastatin, cerivastatin, fluvastatin, lovastatin, pitavastatin (formerly itavastatin), pravastatin, rosuvastatin (formerly visastatin), simvastatin, compactin, mevinolin, mevastatin, velostatin, cerivastatin, synvinolin, or rivastatin (sodium 7-(4-fluorophenyl) 2,6-diisopropyl-5-methoxymethylpyridin-3-yl)3,5-dihydroxy-6-heptanoate) or, in each case, a pharmaceutically acceptable salt thereof. This list is not restrictive and new molecules belonging to this large family are regularly discovered. A statin may be hydrophilic, like pravastatin, or lipophilic, like atorvastatin. Lipophilic statins are believed to better penetrate the tissues. A molecule which is “chemically related or structurally equivalent” to a statin includes molecules whose structure differs from that of any member of the statin family by 2 or fewer substitutions or by modification of chemical bonds. A molecule which is “functionally equivalent” to a statin includes molecules capable of measurable HMG-CoA reductase inhibition. Thus, at least all the molecules capable of competitively inhibiting the enzyme HMG-CoA reductase and called statins possess the required property.

Other features and advantages of the invention will be apparent from the following Detailed Description, the drawings, and the claims.

### BRIEF DESCRIPTION OF THE DRAWINGS

FIG. 1 is the amino acid sequence of human atrogin-1 (Genbank Accession No. AAH24030; SEQ ID NO: 1).

FIG. 2 is the nucleic acid sequence of human atrogin-1 mRNA (Genbank Accession No. BC024030; SEQ ID NO: 2).

FIG. 3 is a diagram showing the location of the N- and C-terminal putative nuclear localization sequences in atrogin-1 (amino acids 62-66 and 267-288, respectively).

FIG. 4A is a set of micrographs showing the morphology of myotubes derived from myoblasts from atrogin-1 knockout mice (−/−) and wildtype mice (+/+) following 24-hour treatment with 0, 0.25, or 1.0 μM lovastatin.

FIG. 4B is a graph showing the percentage of myotube diameter for myotubes derived from atrogin-1 knockout mice (unfilled bars, −/−) and wildtype mice (filled bars, +/+) following 24-hour treatment with 0, 0.25, or 1.0 μM lovastatin compared to myotubes untreated with lovastatin.

FIG. 4C is a graph showing the relative expression level of atrogin-1 mRNA in myotubes derived from atrogin-1 wildtype mice (+/+) following 24-hour treatment with 0, 0.05, 0.25, or 1.0 μM lovastatin compared to myotubes untreated with lovastatin.

FIG. 4D is an immunoblot showing the expression of atrogin-1 protein in myotubes derived from atrogin-1 wildtype (+/+) and atrogin-1 knockout mice (−/−) following 24-hour treatment with 0, 0.05, 0.25, or 1.0 μM lovastatin. The expression level of dynein protein is shown as a control.

FIG. 5A is a set of micrographs showing the morphology of myotubes derived from myoblasts from wildtype mice (+/+) following 24-hour treatment with 0 μM lovastatin and 0 μM mevalonate (control); 1.0 μM lovastatin; 100 μM mevalonate; or 1.0 μM lovastatin and 100 μM mevalonate.

FIG. 5B is a graph showing the percentage of myotube diameter for myotubes derived from atrogin-1 wildtype mice (filled bars, +/+) following 24-hour treatment with 0 μM lovastatin and 0 μM mevalonate (control); 1.0 μM lovastatin; 100 μM mevalonate; or 1.0 μM lovastatin and 100 μM mevalonate compared to untreated myotubes (untreated with lovastatin and mevalonate).

FIG. 5C is a graph showing the relative expression level of atrogin-1 mRNA in myotubes derived from atrogin-1 wildtype mice (+/+) following 24-hour treatment with 0 μM lovastatin and 0 μM mevalonate (control); 1.0 μM lovastatin; 100 μM mevalonate; or

1.0 μM lovastatin and 100 μM mevalonate compared to untreated myotubes (untreated with lovastatin and mevalonate).

FIG. 5D is an immunoblot showing the expression of atrogin-1 protein in myotubes derived from wildtype mice (+/+) following 24-hour treatment with (from left to right) 0 μM lovastatin and 0 μM mevalonate (control); 100 μM mevalonate; 0.5 μM lovastatin; 1.0 μM lovastatin; 100 μM mevalonate and 0.5 μM lovastatin; or 100 μM mevalonate and 1.0 μM lovastatin. The expression level of actin protein is shown as a control.

FIG. 6A is a set of micrographs showing the morphology of myofibers from zebrafish embryos (20 hpf) following 12-hour treatment with 0 μM lovastatin and 0 μM mevalonate (control); 0.05 μM lovastatin; 0.5 μM lovastatin; 100 μM mevalonate; 0.05 μM lovastatin and 100 μM mevalonate; or 0.5 μM lovastatin and 100 μM mevalonate. Side views, anterior views, and left views are shown in the panels from left to right, respectively.

FIG. 6B is a graph showing the relative myofiber diameter for zebrafish myofibers (20 hpf) following 12-hour treatment with 0, 0.05, 0.5, or 1.0 μM lovastatin in the presence (light bars) or absence (dark bars) of 100 μM mevalonate as compared to the myofiber diameter of lovastatin- and mevalonate-untreated myofibers. At least 500 fibers were measured for each treatment condition. The data were graphed as the ratio of mean experimental fiber size +/− standard error of the mean (S.E.M.) to the mean control fiber size +/− S.E.M.

FIG. 6C is an immunoblot showing the expression of atrogin-1 protein in zebrafish myofibers (20 hpf) following 12-hour treatment with (from left to right) 0 μM lovastatin and 0 μM mevalonate (control); 0.5 μM lovastatin; or 0.5 μM lovastatin and 100 μM mevalonate. The expression of glyceraldehyde 3-phosphate dehydrogenase (GAPDH) protein is shown as a control.

FIG. 6D is a graph showing the percentage of damaged zebrafish embryos having class 1, class 2, or class 3 myofiber damage following treatment of wildtype embryos with 0, 0.05, 0.5, or 1 μM lovastatin in the presence (light bars) or absence (dark bars) of 100 μM mevalonate. Class 1 damage include bowing, gap formation, and blocked/disrupted fibers. Class 2 damage includes irregular fibers and diffuse appearance. Class 3 damage are typified by irregular somite boundries. The percentage of embryos displaying specific class defects as a function of lovastatin concentration (+/− mevalonate) are shown. One hundred embryos were quantitated for each treatment.

FIG. 7A is a set of micrographs showing the morphology of myotubes derived from myoblasts from atrogin-1 wildtype mice (+/+) following 24-hour treatment with 0 μM lovastatin, 0 μM geranylgeranol, and 0 μM farnesol (control); 2.5 μM geranylgeranol; 2.5 μM farnesol; 1.0 μM lovastatin; 1.0 μM lovastatin and 2.5 μM geranylgeranol; or 1.0 μM lovastatin and 2.5 μM farnesol.

FIG. 7B is a graph showing the percentage of myotube diameter for myotubes derived from atrogin-1 wildtype mice (+/+) following 24-hour treatment with (from left to right) 0 μM lovastatin, 0 μM geranylgeranol, and 0 μM farnesol (control); 1.0 μM lovastatin; 2.5 μM geranylgeranol; 1.0 μM lovastatin and 2.5 μM geranylgeranol; 2.5 μM farnesol; or 1.0 μM lovastatin and 2.5 μM farnesol compared to the myotube diameter for untreated myotubes.

FIG. 7C is a graph showing the relative expression level of atrogin-1 mRNA in myotubes derived from atrogin-1 wild-type mice (+/+) following 24-hour treatment with 0 μM lovastatin, 0 μM geranylgeranol, and 0 μM farnesol (control); 1.0 μM lovastatin; 2.5 μM geranylgeranol; 1.0 μM lovastatin and 2.5 μM geranylgeranol; 2.5 μM farnesol; or 1.0 μM lovastatin and 2.5 μM farnesol compared to untreated myotubes.

FIG. 7D is an immunoblot showing the expression of atrogin-1 protein in myotubes derived from wildtype mice (+/+) following 24-hour treatment with (from left to right) 0 μM lovastatin, 0 μM geranylgeranol, and 0 μM farnesol (control); 10 μM lovastatin; 2.5 μM geranylgeranol; 10 μM lovastatin and 2.5 μM geranylgeranol; 10 μM lovastatin; or 10 μM lovastatin and 2.5 μM farnesol.

FIG. 8A is a set of micrographs showing the morphology of myofibers from zebrafish embryos (20 hpf) following 12-hour treatment with 0 μM lovastatin, 0 μM geranylgeranyl, and 0 μM farnesol (control; upper left panel); 0.05 μM lovastatin (first row, center panel); 0.5 μM lovastatin (first row, right panel); 0.05 μM lovastatin and 2.5 μM geranylgeranol (second row, left panel); 0.5 μM lovastatin and 2.5 μM geranylgeranol (second row, right panel); 0.05 μM lovastatin and 2.5 μM farnesol (third row, left panel); or 0.5 μM lovastatin and 0.5 μM farnesol (third row, right panel).

FIG. 8B is a graph showing the relative myofiber diameter for zebrafish myofibers (20 hpf) following 12-hour treatment with 0, 0.05, 0.5, or 1 μM lovastatin in the presence of absence (black bars) of either 2.5 μM farnesol (grey bars) or 2.5 μM geranylgeranol (open bars) compared to the diameter of myofibers of untreated zebrafish. The data are graphed as the ratio of mean experimental fiber size +/− S.E.M. to mean control fiber size +/− S.E.M.

FIG. 8C is an immunoblot showing the expression of atrogin-1 protein in zebrafish myofibers (20 hpf) following 12-hour treatment with (from left to right) 0 μM lovastatin, 0 μM geranylgeranol, and 0 μM farnesol (control); 0.5 μM lovastatin; 0.5 μM lovastatin and 10 μM geranylgeranol; 0 μM lovastatin, 0 μM geranylgeranol, and 0 μM farnesol (control); 2.5 μM lovastatin; or 0.5 μM lovastatin and 10 μM farnesol. The expression of GAPDH protein is shown as a control.

FIG. 8D is a graph showing the percentage of damaged zebrafish embryos having class 1, class 2, or class 3 myofiber damage following 12-hour treatment with 0, 0.05, 0.5, or 1 μMM lovastatin in the absence (black bars) or presence of either 2.5 μM geranylgeranol (grey bars) or 2.5 μM farnesol (open bars). The percent of embryos displaying the specific class defects as a function of lovastatin concentration is shown. A total of 100 embryos was quantitated for each treatment.

FIG. 9A is a set of micrographs showing the morphology of myotubes derived from myoblasts from atrogin-1 knockout mice (−/−) and atrogin-1 wildtype mice (+/+) following 24-hour treatment with 0 μM lovastatin, 0 μM N-acetyl-S-farnesyl-L-cysteine (AFC), and 0 μM N-acetyl-S-geranylgeranyl-L-cysteine (AGGC) (control); 250 nM lovastatin; 30 μM AFC; or 30 μM AGGC.

FIG. 9B is a graph showing the percentage of myotube diameter for myotubes derived from atrogin-1 wildtype mice (+/+) following 24-hour treatment with (from left to right) 0 μM lovastatin, 0 μM AFC, and 0 μM AGGC (control); 250 nM lovastatin; 10 μM AFC; 30 μM AFC; 15 μM AGGC; or 30 μM AGGC compared to untreated control myotubes.

FIG. 9C is a graph showing the relative expression level of atrogin-1 mRNA in myotubes derived from atrogin-1 wild-type mice (+/+) following 24-hour treatment with (from left to right) 0 μM AGGC; 15 μM AFC; 30 μM AFC; 15 μM AGGC; or 30 μM AGGC compared to the expression of atrogin-1 in untreated control myotubes. FIG. 9D is an immunoblot showing the expression of atrogin-1 protein in myotubes derived from wild-type mice (+/+) following 24-hour treatment with (from left to right) 0 μM lovastatin, 0 μM AFC, and 0 μM AGGC; 30 μM AFC; or 30 μM AGGC. The expression level of actin protein is shown as a control.

FIG. 10A is a set of micrographs showing the morphology of myotubes derived from myoblasts from atrogin-1 knockout mice (−/−) and atrogin-1 wildtype mice (+/+) following 24-hour treatment with 0 or 10 mM perillyl alcohol.

FIG. 10B is a graph showing the percentage of myotube diameter for myotubes derived from atrogin-1 wildtype mice (+/+; solid bars) and atrogin-1 knockout mice (−/−; open bars) following 24-hour treatment with 0, 0.25, or 1.0 mM perillyl alcohol as compared to untreated control myotubes.

FIG. 10C is a graph showing the relative expression level of atrogin-1 mRNA in myotubes derived from atrogin-1 wildtype mice (+/+) following 24-hour treatment with (from left to right) 0 mM perillyl alcohol; 0.25 mM perillyl alcohol; 1.0 mM perillyl alcohol; or 1 μM lovastatin compared to the level of expression of atrogin-1 in untreated myotubes.

FIG. 10D is an immunoblot showing the expression of atrogin-1 protein in myotubes derived from wildtype mice (+/+) following 24-hour treatment with (from left to right) 0, 0.25, or 1.0 mM perillyl alcohol. The expression level of dynein protein is shown as a control.

FIG. 11A is a set of micrographs showing the morphology of myofibers from zebrafish embryos (20 hpf) following 12-hour treatment with 0 (control), 0.1, 0.2, 0.5, or 1.0 mM perillyl alcohol, or vehicle (0.1% DMSO).

FIG. 11B is a graph showing the relative myofiber diameter for zebrafish myofibers (20 hpf) following 12-hour treatment with (from left to right) 0 mM perillyl alcohol (control); 0.1% DMSO (vehicle); or 0.1, 0.2 , 0.5, or 1.0 mM perillyl alcohol.

FIG. 11C is a graph showing the percentage of damaged zebrafish embryos having class 1, class 2, or class 3 myofiber damage following 12-hour treatment of wildtype embryos with (from left to right) 0 mM perillyl alcohol (control); 0.1% DMSO (vehicle); or 0.1, 0.2, 0.5, or 1.0 mM perillyl alcohol. The number of embryos quantitated for each treatment were 73 for 0.1% DMSO, and 71, 93, 87, 81, and 85 for the perillyl alcohol concentrations of 0, 0.1, 0.2, 0.5, and 1.0 mM, respectively.

FIG. 12A is a set of micrographs showing the morphology of myofibers from control zebrafish embryos (20 hpf) or zebrafish embryos (20 hpf) injected with control morpholinos (control for z-GGTase I or control for z-GGTase II), z-GGTase I morpholino; or z-GGTase II morpholino at a concentration of 0.5 mM or 1.0 mM.

FIG. 12B is a graph showing the relative myofiber diameter for zebrafish myofibers (20 hpf) following injection with control z-GGTase I morpholino; z-GGTase I morpholino; control z-GGTase II morpolino; or z-GGTase II morpholino at a concentration of 4 ng/embryo or 8 ng/embryo as compared to the myofiber diameter from untreated zebrafish embryos.

FIG. 12C is a graph showing the percentage of damaged zebrafish embryos having class 1, class 2, or class 3 myofiber damage following injection with (from left to right) control z-GGTase I morpholino; z-GGTase I morpholino; control z-GGTase II morpholino; or z-GGTase II morpholino at a concentration of 0 ng/embryo, 4 ng/embryo, or 8 ng/embryo. The number of embryos quantitated per treatment are 100, 98, and 102 for controls; 98, 96, and 100 for the z-GGT I knockdowns at the morpholino concentrations of 0, 4, and 8 ng/embryo, respectively; and 75, 88, and 95 for the z-GGT II knockdowns at the morpholino concentrations of 0, 4, and 8 ng/embryo, respectively.

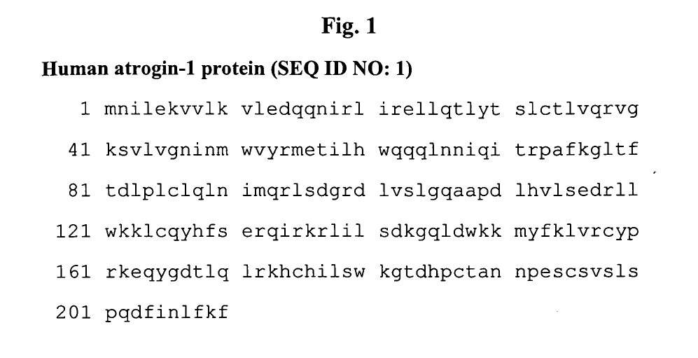

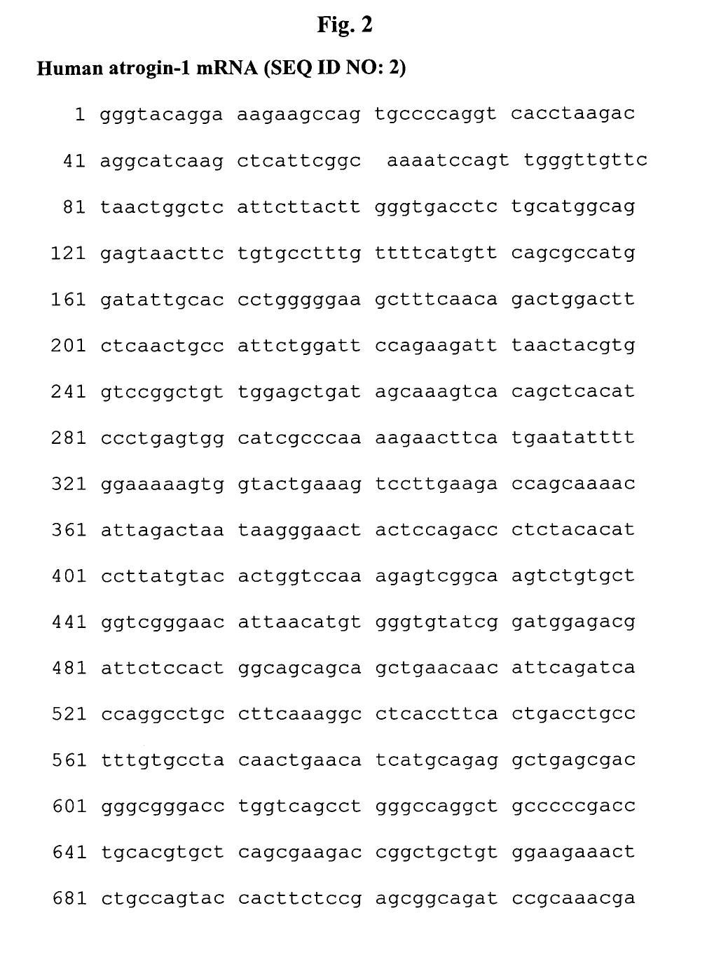

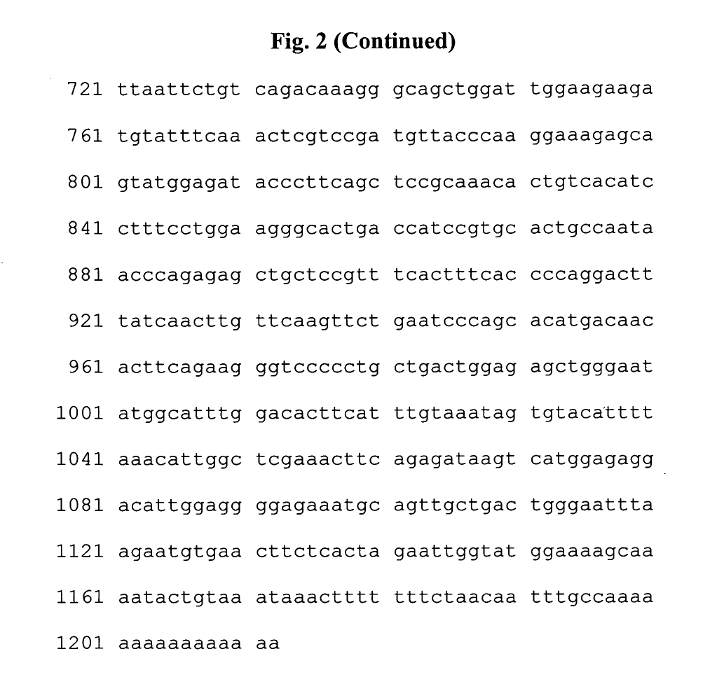

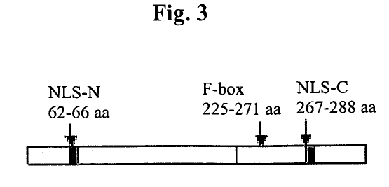

### DETAILED DESCRIPTION

Atrogin-1, an E3 ubiquitn ligase, is upregulated during statin therapy or treatment. Atrogin-1 protein is upregulated in cultured myocytes in the presence of statins in a time- and dose-dependent manner. Furthermore, statins induce myocyte dysfunction as myotubes are improperly formed in the presence of statins. This phenomenon is conserved throughout various species, as murine primary myocyte cell cultures demonstrate the same pattern of a) dose and time dependent upregulation of atrogin-1 in response to statins and b) improper myotube formation in the presence of statins. Zebrafish somite development is also dramatically altered by the presence of statins in the immediate environment (e.g., water). The statin-induced myopathic phenotype in zebrafish can be rescued using a morpholino directed to the atrogin-1 gene. In addition, statin-induced muscle damage did not occur or was vastly diminished in an atrogin-1 knockout mouse model. Taken together, these results support the critical role for atrogin-1 in the pathogenesis of statin-mediated myopathy.

We have discovered that activation of protein geranylgeranylation and geranylgeranylation biosynthetic pathways significantly decreases statin-induced increases in atrogin-1 expression and activity in muscle cells. In view of this discovery, the invention provides methods for treating or reducing the likelihood of developing a statin-induced myopathy that include administering to a subject one or more geranylgeranylation activators.

### Therapeutic Methods

We have discovered that geranylgeranylated proteins and geranylgeranylation biosynthetic pathways in the cell block the activation of atrogin-1 expression and/or activity caused by statin treatment. The invention provide methods for treating or reducing the likelihood of developing a statin-induced myopathy in a subject by administering one or more geranylgeranylation activators (e.g., geranylgeranol). Examples of statin-mediated disorders that can be treated or prevented by the current invention are provided below.

### Statin-Mediated Myopathy

The advent of the HMG-CoA reductase inhibitors, or statins, in the 1980's as highly efficacious agents for the lowering of low-density lipoprotein-cholesterol (LDL-C) revolutionized treatment of hypercholesterolemia, a long established risk factor for premature coronary heart disease. Statins now marketed in the United States include altace (Ramipril), atorvastatin (Lipitor), fluvastatin (Lescol), lovastatin

(Mevacor), pravastatin (Pravachol), simvastatin (Zocor), rosuvastatin, (Crestor), or pitavastatin. Additionally, there are other statins, some in clinical trials, including compactin, mevinolin, mevastatin, velostatin, synvinolin, or rivastatin (sodium 7-(4-fluorophenyl)2,6-diisopropyl-5-methoxymethylpyridin-3-yl)3,5-dihydroxy-6-heptanoate). Statins are well tolerated by most patients but can produce a variety of muscle-related complications or myopathies. The most serious risk of these is myositis with rhabdomyolysis. This risk has been emphasized by the withdrawal of cerivastatin in August 2001 after the drug was associated with approximately 100 rhabdomyolysis-related deaths. Rhabdomyolysis was also a factor in the withdrawal of the antihypertensive drug mibefradil in June 1998 and in the decision by Merck &

Co. to abandon the development of a 160-mg sustained-release simvastatin formulation in the mid-1990s.

Myopathy can refer to any muscular disease, and here we differentiate myalgia as muscle ache or weakness in the absence of elevation in creatine kinase (CK), and myositis as adverse muscular symptoms associated with inflammation with and without increased CK levels. Rhabdomyolysis is a severe form of myositis involving myoglobulinuria, which can engender acute renal failure. Although rhabdomyolysis associated with statin treatment is rare, muscular pain and weakness are more frequent and may affect 7% of patients on statin monotherapy, with myalgia contributing up to 25% of all adverse events associated with statin use (Ucar et al., “HMG-CoA reductase inhibitors and myotoxicity,” Drug Safety 22:441-457, 2000). The effects of subclinical muscular side effects should not be underestimated, however, as they reduce patient compliance with possible discontinuation of therapy, limit physical activity, reduce the quality of life, and most importantly, may ultimately deprive the dyslipidemic patient at high risk for cardiovascular disease of the clinical benefit of statin treatment. Such myopathies become especially pertinent in the context of recent clinical trials, which have validated optimized reduction of morbi-mortality in cardiovascular disease using high-dose statin therapy. This is particularly relevant as increased statin dosage is closely associated with increased risk of muscular side effects. Furthermore, select patient populations require closer surveillance for statin myopathy, as their risk profile is increased. This includes advanced age (>80 years old); small frame; multisystem illness including diabetes; patients in the perioperative period; and concomitant medications. All statin new drug applications (NDAs) suggest that statin myotoxicity is dose dependent with a threshold effect at which risks exceed benefits—especially with regard to cerivastatin.

### Treatment of Statin-Mediated Myopathy

We have discovered that different geranylgeranylation activators prevent atrogin-1 upregulation in myocytes and decrease the amount of statin-induced myocyte dysfunction and improper myotube formation. Any of the geranylgeranylation activators described may be used for the treatment or reduction in the likelihood of developing a statin-mediated myopathy.

Statin-mediated myopathies can be diagnosed using the methods described herein in combination with techniques known in the art (e.g., muscle biopsy and evaluation of atrogin-1 products). Likewise, the therapeutic effectiveness of a geranylgeranylation activator can be measured using the above described in vitro and in vivo assays and methodology. Assays include any of the assays for atrogin-1 biological activity or expression as described herein and in International Patent Application No. PCT/US08/007047 (incorporated in its entirety by reference), wherein a geranylgeranylation activator that reduces or inhibits atrogin-1 biological activity and/or expression is considered an agent useful for the treatment or prevention of a statin-mediated myopathy. Assays of atrogin-1 activity include for example, ubiquitination assays, calcineurin activity assays, substrate binding assays, and nuclear translocation assays. Desirably, geranylgeranylation activators prevent the increase in the expression of atrogin-1 in cells treated with a statin. These assays and evaluation methods can be performed alone, or in combination with other assay techniques evaluating overall muscle health, including CK enzymatic assays.

### Geranylgeranylation Activators

Atrogin-1 levels in myocytes are increased and myocytes are dysfunctional in the presence of a statin. Specifically, myotubes do not form properly and zebrafish somite development is dramatically affected in the presence of statins. We have now discovered that activation of geranylgeranylation proteins and/or geranylgeranylation biosynthetic pathways in a cell effectively blocks statin-induced increases in atrogin-1 expression and/or activity and blocks damage to muscle cells and muscle fibers. The invention therefore features geranylgeranylation activators for treating or reducing the likelihood of developing a statin-mediated myopathy in a subject. The geranylgeranylation activators described below may be used or administered in combination with one or more (e.g., 1, 2, 3, 4, 5, 6, 7, 8, 9, or 10) of the atrogin-1 inhibitor compounds previously described in International Patent Application No. PCT/US08/007047 (herein incorporated by reference in its entirety). Preferred geranylgeranylation activators will reduce or inhibit atrogin-1 biological activity and/or expression levels by at least 10%, 25%, 30%, 40%, 50%, 60%, 70%, 80%, 90%, 95%, 96%, 97%, 98%, 99%, or more. Desirably, the geranylgeranylation activator can reduce or inhibit myocyte and/or myotube dysfunction, somite developmental irregularities, increased atrogin-1-specific ubiquitination of muscle proteins, atrogin-1-specific substrate binding of muscle proteins, and symptoms of a myopathy or statin-mediated myopathy, including myalgias, myalagia with associated creatine kinase elevations, myositis, or rhabdomyolysis by at least 10%, 20%, 25%, 30%, 40%, 50%, 60%, 70%, 80%, 90%, 95%, 96%, 97%, 98%, 99%, or more. Preferred geranylgeranylation activators will also block a statin-induced increase in atrogin-1 expression levels by at least 5%, 10%, 15%, 20%, 25%, 30%, 35%, 40%, 45%, 50%, 60%, 70%, 80%, 90%, 95%, 96%, 97%, 98%, 99%, or more.

Desirable geranylgeranylation activators may also mediate an increase (e.g., at least 5%, 10%, 15%, 20%, 25%, 30%, 40%, 50%, 60%, 70%, 80%, 90%, or 100% increase) in the geranylgeranylation and/or biological activity of one or more gernaylgeranylated proteins, including, for example, small GTPases (e.g., rac, rho, rheb, and rap1). A geranylgeranylation activator may also increase (e.g., at least 5%, 10%, 15%, 20%, 25%, 30%, 35%, 40%, 45%, 50%, 60%, 70%, 80%, 90%, 95%, or 100%) the expression and/or biological activity of one or more (e.g., 1, 2, 3, 4, 5, 6, 7, or 8) enzymes or proteins involved in geranylgeranylation biosynthetic pathways, including but not limited to, mevalonate kinase, phosphomevalonate kinase, diphosphomevalonate decarboxylase, isopentenyl-diphosphate delta-isomerase, famesyl diphosphate synthase, geranylgeranyl diphosphate synthase 1, farnesyltransferase, protein geranylgeranyltransferase, rab geranylgeranyltransferase, and isoprenylcysteine carboxyl methyltransferase. The increase in the activity and/or expression of one or more of the enzymes or proteins involved in geranylgeranylation biosynethic pathways in a cell treated with an geranylgeranylation activator may be compared to the expression and/or activity of the same enzymes or proteins in an untreated cell.

Desirable geranylgeranylation activators will mediate an increase (e.g., at least 5%, 10%, 15%, 20%, 25%, 30%, 40%, 50%, 60%, 70%, 80%, 90%, or 100% increase) in the level of one or more metabolites or intermediates (e.g., 1, 2, 3, 4, 5, 6, 7, 8, or 9) in the geranylgeranylation biosynthetic pathways in the cell (e.g., mevalonate, phosphomevalonate, 5-pyrophosphomevalonate, isopentenyl pyrophosphate, dimethylallyl pyrophosphate, famesyl diphosphate, geranyl diphosphate, geranylgeranyl pyrophosphate, or geranylgeranol). Specific examples of geranylgeranylation inhibitors are described below.

### Small Molecules

A geranylgeranylation activator may be a small molecule or a metabolite of a geranylgeranylation biosynthetic pathway or a small molecule that activates the expression and/or activity of one or more of the enzymes in a geranylgeranylation biosynthetic pathway. Examples of geranylgeranylation activators that are metabolites include mevalonate, phosphomevalonate, 5-pyrophosphomevalonate, isopentenyl pyrophosphate, dimethylallyl pyrophosphate, farnesyl diphosphate, geranyl diphosphate, geranylgeranyl pyrophosphate, or geranylgeranol, or compounds which will breakdown to yield these molecules (e.g., a salt comprising one of these molecules).

Additional small molecules that are desirable geranylgeranylation activators may be identified in screening assays. In such screening assays, a cell is administered a candidate small molecule and one or more of the following are measured: the expression and/or activity of atrogin-1 (in the presence or absence of a statin), the expression and/or activity of one or more enzymes in a geranylgeranylation biosynthetic pathway (e.g., mevalonate kinase, phosphomevalonate kinase, diphosphomevalonate decarboxylase, isopentenyl-diphosphate delta-isomerase, farnesyl diphosphate synthase, geranylgeranyl diphosphate synthase 1, farnesyltransferase, protein geranylgeranyltransferase, rab geranylgeranyltransferase, and isoprenylcysteine carboxyl methyltransferase), the level of protein geranylgeranylation, and/or the expression and/or activity of one or more geranylgeranylated proteins in the cell (e.g., one or more small GTPases, such as rac, rho, rheb, and rap1). One or more of the above activities may be measured in a multi-well screening assay using any type of cell (e.g., a myocyte, a myoblast, or a cardiomyocyte).

Large libraries of both natural product or synthetic (or semi-synthetic) extracts and chemical libraries are available in the art. For example, synthetic compound libraries are commercially available from Brandon Associates (Merrimack, N.H.) Aldrich Chemical (Milwaukee, Wis.), and ChemBridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries may be produced, if desired, according to methods known in the art, e.g., by standard extraction and fractionation methods. Furthermore, if desired, any library or compound may be readily modified using standard chemical, physical, or biochemical methods.

One or more small molecule geranylgeranylation activators may be administered to a subject or packaged in a kit with one or more additional atrogin-1 inhibitors (e.g., one or more atrogin-1 inhibitor compounds described in International Patent Application No. PCT/US08/007047).

### Nucleic Acids and Polypeptides

A geranylgeranylation activator may also be a nucleic acid or polypeptide. Examples of geranylgeranylation activators are nucleic acids containing a nucleic acid sequence at least 85% identical (e.g., at least 90%, 95%, 96%, 97%, 98%, 99%, or 100%) to a sequence encoding one or more enzymes in the geranylgeranylation biosynthetic pathway, including but not limited to, mevalonate kinase (NCBI Accession No. NP_{—}000422), phosphomevalonate kinase (NCBI Accession No. NP_{—}006547), diphosphomevalonate decarboxylase (NCBI Accession No. NP_{—}002452), isopentenyl-diphosphate delta-isomerase (NCBI Accession Nos. NP_004499 and NP_{—}150286), farnesyl diphosphate synthase (also known as farnesyl pyrophosphate synthetase, dimethylallyltranstransferase, and geranyltranstransferase; NCBI Accession No. NP_{—}001129293), geranylgeranyl diphosphate synthase 1 (NCBI Accession No. NP_{—}001032354), farnesyltransferase CAAX box alpha (NCBI Accession No. NP_{—}002018), farnesyltransferase CAAX box beta (NCBI Accession No. NP_{—}002019), protein geranylgeranyltransferase type 1 beta subunit (NCBI Accession No. NP_{—}005014.2), rab geranylgeranyltransferase alpha subunit (NCBI Accession No. NP_{—}878256.1), rab geranylgeranyltransferase beta subunit (NCBI Accession No. NP_{—}004573.2), and isoprenylcysteine carboxyl methyltransferase (NCBI Accession No. NP_{—}036537.1), or a active fragment thereof (e.g., a fragment encoding an protein with at least 60%, 70%, 80%, 90%, 95%, 99%, or 100% of the actvitiy of the full-length enzyme). Activity assays for the enzymes in a geranylgeranylation biosynthetic pathway are known in the art. Active fragments may also be identified using any of the assays described herein for the identification of a geranylgeranylation activator (e.g., atrogin-1 expression and/or activity assays).

A geranylgeranylation activator may also be a polypeptide including a polypeptide containing a sequence at least 80% identical (e.g., at least 90%, 95%, 96%, 97%, 98%, 99%, or 100%) to a sequence encoding one more enzymes in the geranylgeranylation biosynethetic pathway, including but not limited to, mevalonate kinase (NCBI Accession No. NP_{—}000422), phosphomevalonate kinase (NCBI Accession No. NP_{—}006547), diphosphomevalonate decarboxylase (NCBI Accession No. NP_{—}002452), isopentenyl-diphosphate delta-isomerase (NCBI Accession Nos. NP_{—}004499 and NP_{—}150286), farnesyl diphosphate synthase (NCBI Accession No. NP_{—}001129293), geranylgeranyl diphosphate synthase 1 (NCBI Accession No. NP_{—}001032354), farnesyltransferase CAAX box alpha (NCBI Accession No. NP_{—}002018), farnesyltransferase CAAX box beta (NCBI Accession No. NP_{—}002019), protein geranylgeranyltransferase type 1 beta subunit (NCBI Accession No. NP_{—}005014.2), rab geranylgeranyltransferase alpha subunit (NCBI Accession No. NP_{—}878256.1), rab geranylgeranyltransferase beta subunit (NCBI Accession No. NP_{—}004573.2), and isoprenylcysteine carboxyl methyltransferase (NCBI Accession No. NP_{—}036537.1), or a fragment thereof (e.g., a fragment having at least 60%, 70%, 80%, 90%, 95%, 99%, or 100% of the full-length enzyme). As stated above, activity assays for the enzymes in a geranylgeranylation biosynthetic pathway are known in the art. Active fragments may also be identified using any of the assays described herein for the identification of a geranylgeranylation activator (e.g., atrogin-1 expression assays).

### Therapeutic Formulations and Kits

Statin compounds for use in the methods are pharmaceutical formulations of the HMG-CoA reductase inhibitors are understood to be those active agents which may be used to lower the lipid levels including cholesterol in blood. The class of HMG-CoA reductase inhibitors comprises compounds having differing structural features. For example, HMG-CoA reductase inhibitors include: atorvastatin, cerivastatin, fluvastatin, lovastatin, pitavastatin (formerly itavastatin), pravastatin, rosuvastatin, and simvastatin, or, in each case, a pharmaceutically acceptable salt thereof (e.g., a calcium salt). Preferred HMG-CoA reductase inhibitors are those agents which have been marketed as lipid lowering compounds, most preferred is fluvastatin, atorvastatin, pitavastatin or simvastatin, or a pharmaceutically acceptable salt thereof.

The dosage of the active compound can depend on a variety of factors, such as mode of administration, homeothermic species, age and/or individual condition. Statins may be administered at a dosage of generally between about 1 and about 500 mg/day, more preferably from about 1 to about 40, 50, 60, 70 or 80 mg/day, advantageously from about 20 to about 40 mg per day. For example, tablets or capsules comprising, e.g., from about 5 mg to about 120 mg, preferably, when using fluvastatin, for example, 20 mg, 40 mg, or 80 mg (equivalent to the free acid) of fluvastatin, for example, administered once a day.

The invention includes the use of one or more geranylgeranylation activators (e.g., 1, 2, 3, 4, 5, 6, 7, or 8) to treat, prevent, or reduce a statin-mediated myopathy in a subject. The geranylgeranylation activator can be administered at anytime, for example, after diagnosis or detection of a statin-mediated myopathy, or for prevention of a statin-mediated myopathy in subjects that have not yet been diagnosed with a statin-mediated myopathy but are at risk of developing such a disorder, or after a risk of developing a statin-mediated myopathy is determined. A geranylgeranylation activator may also be administered simultaneously with a statin. A geranylgeranylation activator of the invention may be formulated with a pharmaceutically-acceptable diluent, carrier, or excipient, in unit dosage form. Conventional pharmaceutical practice may be employed to provide suitable formulations or compositions to administer a geranylgeranylation activator of the invention to patients suffering from a statin-mediated myopathy. Administration may begin before the patient is symptomatic. The geranylgeranylation activator of the present invention can be formulated and administered in a variety of ways, e.g., those routes known for specific indications, including, but not limited to, topically, orally, subcutaneously, bronchioscopic injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. The geranylgernaylation activator can be in the form of a pill, tablet, capsule, liquid, or sustained release tablet for oral administration; or a liquid for intravenous administration, subcutaneous administration, or injection; for intranasal formulations, in the form of powders, nasal drops, or aerosols; or a polymer or other sustained-release vehicle for local administration.

The invention also includes the use of one or more geranylgeranylation activators (e.g., 1, 2, 3, 4, 5, 6, or 7) to treat, prevent, or reduce a statin-mediated myopathy in a biological sample derived from a subject (e.g., treatment of a biological sample ex vivo) using any means of administration and formulation described herein. The biological sample to be treated ex vivo may include any biological fluid (e.g., blood, serum, plasma, or cerebrospinal fluid), cell (e.g., a myocyte), or tissue (e.g., muscle tissue) from a subject that has a statin-mediated myopathy or the propensity to develop a statin-induced myopathy. The biological sample treated ex vivo with the geranylgeranylation activator may be reintroduced back into the original subject or into a different subject. The ex vivo treatment of a biological sample with a geranylgeranylation activator, as described herein, may be repeated in an individual subject (e.g., at least once, twice, three times, four times, or at least ten times). Additionally, ex vivo treatment of a biological sample derived from a subject with a geranylgeranylation activator, as described herein, may be repeated at regular intervals (non-limiting examples include daily, weekly, monthly, twice a month, three times a month, four times a month, bi-monthly, once a year, twice a year, three times a year, four times a year, five times a year, six times a year, seven times a year, eight times a year, nine times a year, ten times a year, eleven times a year, and twelve times a year).

Therapeutic formulations are prepared using standard methods known in the art by mixing the active ingredient having the desired degree of purity with optional physiologically acceptable carriers, excipients or stabilizers (Remington's Pharmaceutical Sciences (20th edition), Ed. A. Gennaro, 2000, Lippincott, Williams & Wilkins, Philadelphia, Pa.), in the form of lyophilized formulations or aqueous solutions. Acceptable carriers, include saline, or buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG. Optionally, but preferably, the formulation contains a pharmaceutically acceptable salt, preferably sodium chloride, and preferably at about physiological concentrations. The formulation may also contain the geranylgeranylation activator in the form of a calcium salt. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents.

Compositions of the invention may contain one or more geranylgeranylation activator(s) with one or more (e.g., 1, 2, 3, 4, 5, 6, 7, 8, 9, or 10) of a statin compound (e.g., atorvastatin, cerivastatin, fluvastatin, lovastatin, pitavastatin (formerly itavastatin), pravastatin, rosuvastatin, and simvastatin), a COX-2 inhibitor (e.g., rofecoxib, celecoxib, valdecoxib, and lumiracoxib), a non-steroidal anti-inflammatory molecule (NSAID; e.g., naproxen sodium, diclofenac sodium, diclofenac potassium, aspirin, sulindac, diflusinal, piroxicam, indomethacin, ibuprofen, nabumetone, choline magnesium trisalicylate, sodium salicylate, salicylsalicyclic acid, fenoprofen, flurbiprofen, ketoprofen, meclofenamate sodium, meloxican, oxaprozin, sulindac, and tolmetin), a DMARD (e.g., methotrexate, leflunomide, minocycline, auranofin, gold sodium thiomalate, aurothioglucose, and azathioprine), a biologic (e.g., alafacept, infliximab, adelimumab, efalizumab, etanercept, and CDP-870), a calcineurin inhibitor (e.g., cyclosporine A, tacrolimus, and pimecrolimus), and/or a corticosteroid (e.g., prenisolone, prednisone, budesonide, fluticasone propionate, triamcinolone acetonide, hydrocortisone, and methylprednisolone). Such co-formulated compositions may include one or more geranylgeranylation activator(s) and one or more statin compound, COX-2 inhibitor, NSAID, DMARD, biologic, calcineurin inhibitor, or corticosteroid formulated together in the same pill, capsule, liquid, etc. By using different formulation strategies for the different agents, the pharmokinetic profiles of each agent can be suitably matched.

The compositions of the invention may be formulated as a multi-layer (e.g., bi-layer, three-layer, or four-layer) tablet. An example of a bilayer tablet contains a layer containing one or more (e.g., 1, 2, 3, 4, or 5) geranylgeranylation activators and an additional layer containing one or more of a statin compound, COX-2 inhibitor, NSAID, DMARD, biologic, calcineurin inhibitor, and/or corticosteroid. A bilayer tablet can be formulated in which different custom granulations are made for each agent of the combination and the agents are compressed on a bi-layer press to form a single tablet. For example, 1 to 25 mg, 20 to 50 mg, 20 to 100 mg, or 100 to 200 mg of one or more geranylgeranylation activators, formulated for a controlled release that results in a t_{1/2} of 10 to 20 hours may be combined in the same tablet with 1 to 40 mg, 10 to 50 mg, 20 to 60 mg, 50 to 100 mg, or 100 to 200 mg of a second agent (e.g., a statin compound), which is formulated such that the t_{1/2} approximates that of the one or more geranylgeranylation activator(s). Examples of extended-release formulations, including those used in bilayer tablets, can be found in U.S. Pat. No. 6,548,084 (herein incorporated by reference). In addition to controlling the rate of release in vivo, an enteric or delayed release coat may be included that delays the start of drug release such that the Tₘₐₓ of the one or more geranylgeranylation activator(s) approximates that of the second agent (e.g., a statin compound).

Administration of one or more geranylgeranylation activators and a second agent (e.g., statin compound, COX-2 inhibitor, NSAID, DMARD, biologic, calcineurin inhibitor, and corticosteroid) for controlled release is useful where the one or more geranylgeranylation activator and/or the second agent, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount of that is therapeutically effective for the treatment of myopathy (e.g., statin-induced myopathy). Accordingly, a sustained release formulation may be used to avoid frequent dosing that may be required in order to sustain the plasma levels of the agents at a therapeutic level. For example, in preferable oral pharmaceutical compositions of the invention, half-life and mean residency times from 10 to 20 hours for one or both of the geranylgeranylation activator and the second agent are desired.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the one or more geranylgeranylation activator(s) and/or the second agent are released at period intervals, the release could be simultaneous, or a delayed release of one of the agents of the combination can be affected, when the early release of one particular agent (e.g., a geranylgeranylation activator) is preferred over the other.

Controlled release formulations may include a degradable or nondegradable polymer, hydrogel, organogel, or other physical construct that modifies the bioabsorption, half-life or biodegradation of the agent. The controlled release formulation can be a material that is painted or otherwise applied onto the afflicted site, either internally or externally. In one example, the invention provides a biodegradable bolus or implant that is surgically inserted at or near a site of interest (for example, proximal to a muscle tissue).

Hydrogels can be used in controlled release formulations for the compositions of the present invention. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylactic acid, polycaprolactone, polyorthoesters, polyanhydrides or polypeptides. The entire network can be gelled into a biodegradable network that can be used to entrap and homogeneously disperse geranylgeranylation activator/second agent combinations of the invention for delivery at a controlled rate.

Chitosan and mixtures of chitosan with carboxymethylcellulose sodium (CMC-Na) have been used as vehicles for the sustained release of drugs, as described by Inouye et al., Drug Design and Delivery 1: 297-305, 1987. Mixtures of these compounds and agents of the geranylgeranylation activator/second agent combinations of the invention, when compressed under 200 kg/cm², form a tablet from which the active agent is slowly released upon administration to a subject. The release profile can be changed by varying the ratios of chitosan, CMC-Na, and active agent(s). The tablets can also contain other additives, including lactose, CaHPO₄ dihydrate, sucrose, crystalline cellulose, or croscarmellose sodium.

Baichwal, in U.S. Pat. No. 6,245,356, describes a sustained release oral solid dosage forms that includes agglomerated particles of a therapeutically active medicament in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well-known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the compositions of the invention, Baichwal and Staniforth in U.S. Pat. No. 5,135,757 describe a free-flowing slow release granulation for use as a pharmaceutical excipient that includes from about 20 to about 70 percent or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and from about 30 to about 80 percent by weight of an inert pharmaceutical filler (such as, for example, lactose, dextrose, sucrose, sorbitol, xylitol, fructose or mixtures thereof). After mixing the excipient with an geranylgeranylation activator/second agent combination, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the combinations of the invention, Shell, in U.S. Pat. No. 5,007,790, describe sustained-release oral drug-dosage forms that release a drug in solution at a rate controlled by the solubility of the drug. The dosage form comprises a tablet or capsule that includes a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles, assuring that drug reaches the stomach in the solution state which is less injurious to the stomach than solid-state drug. The programmed eventual dissolution of the polymer depends upon the nature of the polymer and the degree of crosslinking. The polymer is nonfibrillar and substantially water soluble in its uncrosslinked state, and the degree of crosslinking is sufficient to enable the polymer to remain insoluble for the desired time period, normally at least from about 4 hours to 8 hours up to 12 hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of the geranylgeranylation activator/second agent combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500 to 1000 μm size range.

Slow-release formulations can include a coating which is not readily water-soluble but which is slowly attacked and removed by water, or through which water can slowly permeate. Thus, for example, the geranylgeranylation activator/second agent combinations of the invention can be spray-coated with a solution of a binder under continuously fluidizing conditions, such as describe by Kitamori et al., U.S. Pat. No. 4,036,948. Examples of water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, organic solvent-soluble binders, such as cellulose derivatives (e.g., cellulose acetate phthalate, hydroxypropylmethyl-cellulose phthalate, ethylcellulose).

Compositions of the invention with sustained release properties can also be formulated by spray drying techniques. In one example, as described by Espositio et al., Pharm. Dev. Technol. 5: 267-78, 2000, prednisolone was encapsulated in methyacrylate microparticles (Eudragit RS) using a Mini Spray Dryer, model 190 (Buchi, Laboratorium Technik AG, Flawil, Germany). Optimal conditions for microparticle formation were found to be a feed (pump) rate of 0.5 mL/min of a solution containing 50 mg prednisolone in 10 mL of acetonitrile, a flow rate of nebulized air of 600 L/hr, dry air temperature heating at 80° C., and a flow rate of aspirated drying air of 28 m³/hr.

Yet another form of sustained release geranylgeranylation activator/second agent combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, for example, Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

The compositions of the invention may also be formulated for parenteral administration. For example, the geranylgeranylation activator may be formulated in a unit dosage injectable form (solution, suspension, emulsion) in association with a pharmaceutically acceptable parenteral vehicle. Such vehicles are inherently nontoxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of molecules, polypeptides, and fragments available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the molecule or polypeptide in a suitable delivery vehicle (e.g., polymeric microparticles or implantable devices) may increase the efficiency of delivery, particularly for oral delivery.

As described above, the dosage of the geranylgeranylation activator will depend on other clinical factors such as weight and condition of the subject and the route of administration of the compound. For treating subjects, between approximately 0.01 mg/kg to 500 mg/kg body weight of the geranylgeranylation activator can be administered. A more preferable range is 0.01 mg/kg to 50 mg/kg body weight with the most preferable range being from 1 mg/kg to 25 mg/kg body weight. Depending upon the half-life of the geranylgeranylation activator in the particular subject, the geranylgeranylation activator can be administered between several times per day to once a week. The methods of the present invention provide for single as well as multiple administrations, given either simultaneously or over an extended period of time.

Alternatively, a polynucleotide containing a nucleic acid sequence encoding a geranylgeranylation activator (e.g., an mRNA encoding an enzyme in the geranylgeranylation pathway) can be delivered to the appropriate cells in the subject. Expression of the coding sequence can be directed to any cell in the body of the subject, preferably a myocyte. This can be achieved, for example, through the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome-mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of a geranylgeranylation activator.

Alternatively, tissue specific targeting can be achieved by the use of tissue- or cell-specific transcriptional regulatory elements which are known in the art (e.g., myocyte-specific promoters or enhancers). Delivery of “naked DNA” (i.e., without a delivery vehicle) to an intramuscular, intradermal, or subcutaneous site is another means to achieve in vivo expression.

Gene delivery using viral vectors such as adenoviral, retroviral, lentiviral, or adeno-asociated viral vectors can also be used. Numerous vectors useful for this purpose are generally known and have been described. In the relevant polynucleotides (e.g., expression vectors), the nucleic acid sequence encoding one or more geranylgeranylation activators (e.g., a nucleic acid encoding one or more of a protein at least 85% identical to mevalonate kinase, phosphomevalonate kinase, diphosphomevalonate decarboxylase, isopentenyl-diphosphate delta-isomerase, farnesyl diphosphate synthase, geranylgeranyl diphosphate synthase 1, farnesyltransferase, protein geranylgeranyltransferase, rab geranylgeranyltransferase, and isoprenylcysteine carboxyl methyltransferase) is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety.

An ex vivo strategy can also be used for therapeutic applications. Ex vivo strategies involve transfecting or transducing cells obtained from the subject with a polynucleotide encoding a geranylgeranylation activator. The transfected or transduced cells are then returned to the subject. Such cells act as a source of the geranylgeranylation activator for as long as they survive in the subject.

Geranylgeranylation activators for use in the present invention may also be modified in a way to form a chimeric molecule comprising a geranylgeranylation activator fused to another, heterologous polypeptide or amino acid sequence, such as an Fc sequence for stability.

The geranylgeranylation activator can be packaged alone or in combination with other therapeutic compounds as a kit (e.g., with a statin compound or one or more additional atrogin-1 inhibitor compounds). Non-limiting examples include kits that contain, for example, two pills, a powder, a suppository and a liquid in a vial, or two topical creams. Desirably, a kit contains both a geranylgeranylation activator and a statin.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Combination therapies of the invention include this sequential administration, as well as administration of these therapeutic agents, in a substantially simultaneous manner. Substantially simultaneous administration can be accomplished, for example, by administering to the subject a geranylgeranylation activator and a statin in multiple capsules or injections. The components of the combination therapies, as noted above, can be administered by the same route or by different routes. For example, a statin compound and a geranylgeranylation activator may both be administered in the same way (e.g., via oral administration). In different embodiments, a statin compound may be administered by orally, while the other geranylgeranylation activators may be administered intramuscularly, subcutaneously, topically or all therapeutic agents may be administered orally or all therapeutic agents may be administered by intravenous injection. The temporal sequence and or route of administration in which the therapeutic agents may depend upon the status of the patient. For example a patient at risk for developing a statin-mediated myopathy may begin receiving an administration or multiple administrations of a geranylgeranylation activator prior to administration of a statin compound, simultaneously to a statin compound, or consequent to administration of a statin compound. A patient diagnosed with a statin-mediated myopathy may, for example, terminate administration of a statin compound, or receive administration of a different statin compound while beginning or maintaining administration of a geranylgeranylation activator. Likewise, monitoring a patient undergoing treatment for a statin mediated myopathy would likely dictate the temporal sequence and/or route of administration based on the efficacy of treatment as established by the clinician.

While the approaches to be utilized in the invention have been described above, the techniques that are utilized are described in greater detail below. These examples are provided to illustrate the invention, and should not be construed as limiting.

### EXAMPLES

### Example 1

### Role of Atrogin-1 in Muscle Damage

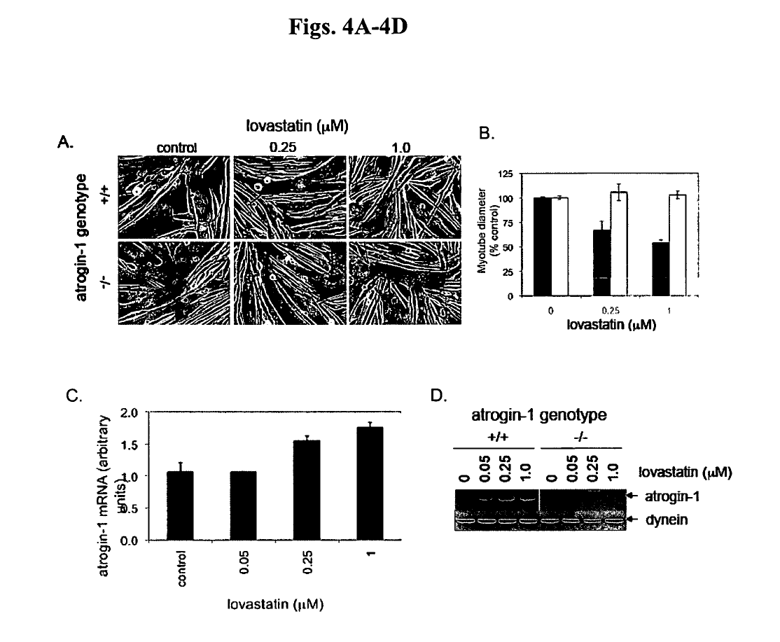

Myotubes derived from atrogin-1 knockout (−/−) and wildtype mice were administered 0.5 to 1.0 μM lovastatin for 24 hours. Administration of lovastatin led to a dose-dependent reduction in myotube diameter in wildtype mice. In contrast, administration of lovastatin to atrogin-1 knockout cultures were resistant to lovastatin-induced reduction in myotube diameter (FIGS. 4A and 4B). Concordant with the observed reduction in myotube diameter, administration of lovastatin to wildtype cultures led to a dose-dependent increase in atrogin-1 mRNA and protein levels (FIGS. 4C and 4D, respectively). These results are consistent with previous results (Hanai et al., J. Clin. Invest. 117:3940-3951, 2007). It is further noted that the concentrations of lovastatin used in these experiments reflect the serum lovastatin concentrations typically observed in patients (Pan et al., J. Clin. Pharmacol. 30:1128-1135, 1990; Holstein et al., Cancer Chemother. Pharmacol. 57:155-164, 2006).

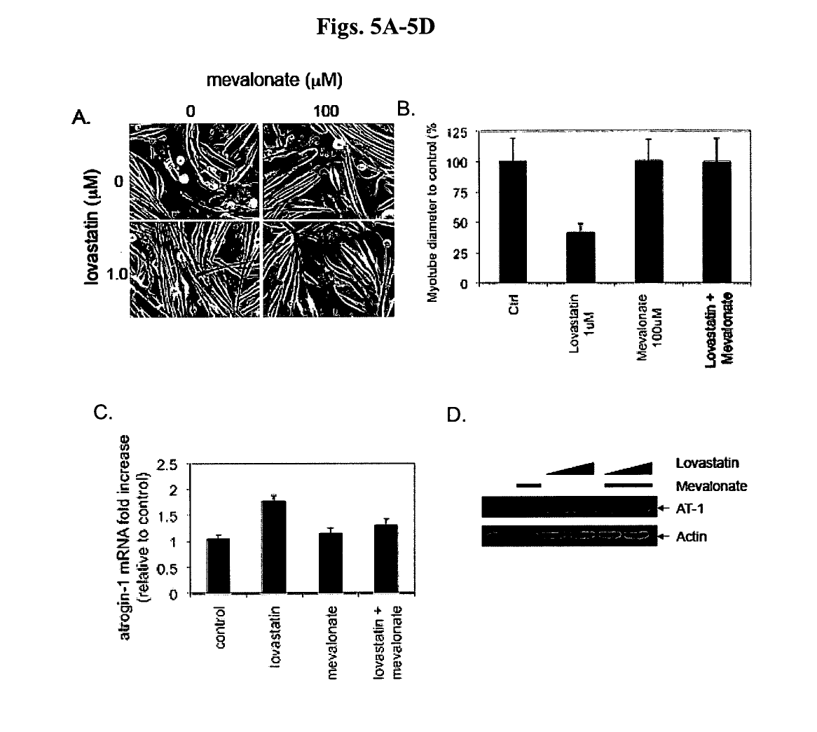

Additional experiments were performed to ensure that the effect of lovastatin was due to inhibition of HMG CoA reductase rather than a non-specific effect in the cells. In these experiments, wildtype mouse myotubes were treated with lovastatin in combination with the product of HMG CoA reductase, mevalonate. Mevalonate (100 μM) treatment for 24-hours had no effect on the muscle cultures when administered alone, but completely prevented the reduction of myotube diameter, and atrogin-1 mRNA and protein induction caused by 24-hour treatment with lovastatin (1 μM) (FIGS. 5A-5D).

### Experimental Methods

Cell Culture: Lovastatin (>98% purity) (mevinolin, Sigma) was prepared as a 50 mM stock solution in DMSO as reagent vehicle, further diluted in DMSO and added into the medium. The final volume DMSO in medium was not more than 0.125%, at which there is no obvious cytotoxicity. Equal volume of reagent vehicle was used for all experiment and regent vehicle serviced as controls. Each experiment was performed for at least three times. Primary mouse fibroblasts from atrogin-1 knockout mice (−/−; Regeneron) were isolated as follows: muscle was removed from the hind limbs of two-week old mice. After treatment with 0.1% collagenase D and Dispase II (Roche), the isolated cells were plated on collagen (Type 1, Roche)-coated dishes. Myoblasts were subsequently enriched and cultured in F-10 nutrient medium with 20% fetal calf serum and 2.5 ng/mL bFGF (Invitrogen). Myotubes were induced in differentiation medium. All media contained 1× Primocin (InvivoGen). The cultures were maintained at 37° C., under 5% and 8% CO₂ air-humidified atomosphere for myoblasts and myotubes, respectively. Cultures were ready to use in assays on day 2 in differentiation medium, when the myotubes had formed and were contracting.

Myotube fiber size: Size was quantified by measuring a total of 200 tube diameters as described in Sandri et al. (Cell 117:399-412, 2004). Briefly, muscle fiber size from four random fields at 100-fold magnification was measured using IMAGE software (Scion). All data were expressed as the mean +/− S.E.M.

Quantitative PCR: Atrogin-1 mRNA levels were determined by real-time PCR using the Applied Biosystems® 7500 real-time PCR analyzer according to the method recently described by others (Okuno et al., Blood 100:4420-4426, 2002, and Yang et al., J. Cell Biochem. 94:1058-1067, 2005). Multiplexed amplification reactions were performed using 18S rRNA as an endogenous control (18S rRNA primers/VIC-labeled probe Applied Biosystems #4310893E) using the TaqMan One Step PCR Master Mix reagents Kit (#4309169, Applied Biosystems). The following settings were used: Stage 1 (reverse transcription): 48° C. for 30 min; Stage 2 (denaturation): 95° C. for 10 min; and Stage 3 (PCR): 95° C. for 15 sec and 60° C. for 60 sec for 40 cycles. The sequences of the forward, reverse, and double-labeled oligonucleotides for atrogin-1 were: forward 5′-CTT TCA ACA GAC TGG ACT TCT CGA -3′ (SEQ ID NO: 3); reverse 5′-CAG CTC CAA CAG CCT TAC TAC GT-3′ (SEQ ID NO: 4); and TaqMan® probe: 5′- FAM-TGC CAT CCT GGA TTC CAG AAG ATT CAA C-TAMRA-3′ (SEQ ID NO: 5). Fluorescence data were analyzed by SDS 1.7 software (Applied Biosystems). The Ct (threshold cycle) values for each reaction were transferred to a Microsoft Excel spreadsheet and calculation of relative gene expression was performed from this data according to published algorithms (TaqMan Cytokine Gene Expression Plate 1 protocol, Applied Biosystems). All RNA samples were analyzed in triplicate, with the mean value used in subsequent analyses.

Western blotting: Cultured cells after treatment were collected at specific times and solubilized in RIPA lysis buffer (50 mM Tris-HCl, pH 7.4, 150 mM NaCl 1% NP-40, 0.5% sodium deoxycholate, 0.1% SDS (Boston Bioproducts)), protease (Roche) and phosphatase (Sigma) inhibitor cocktail). Proteins were separated by SDS-PAGE, transferred to PVDF membranes and visualized by Western blotting using alkaline phosphatase-based CDP-star chemiluminescent detection according to the manufacturer's protocol (Applied Biosystems). Polyclonal anti-atrogin-1 antibody was used as described in Bdolah et al. (Am. J. Physiol. Reg. Integr. Comp. Physiol. 292:R971-R976, 2007). Anti-actin and dynein antibodies were from Santa Cruz Biotechnology.

### Example 2

### Mevalonate Rescues Statin-Induced Muscle Damage

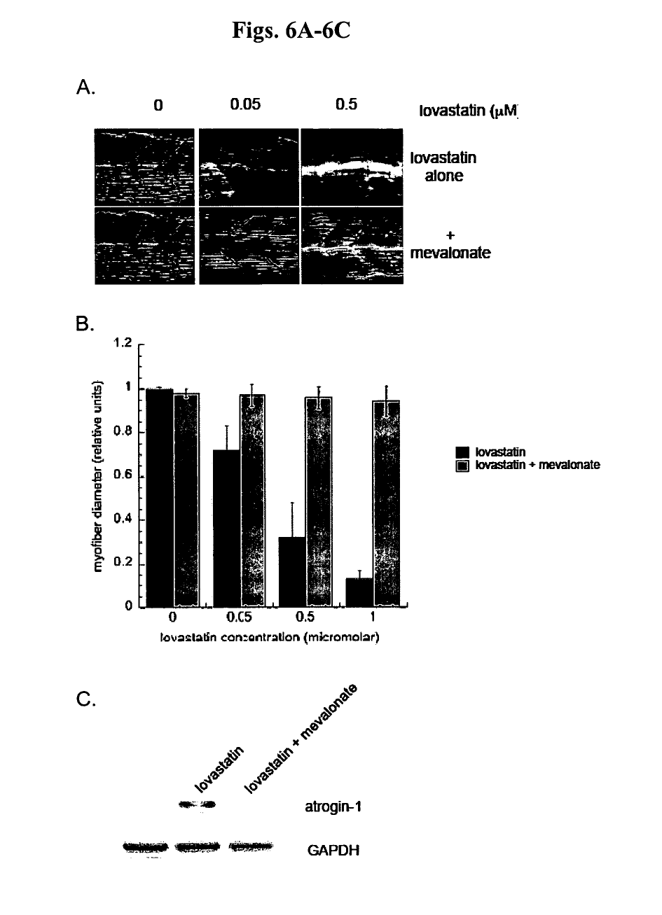

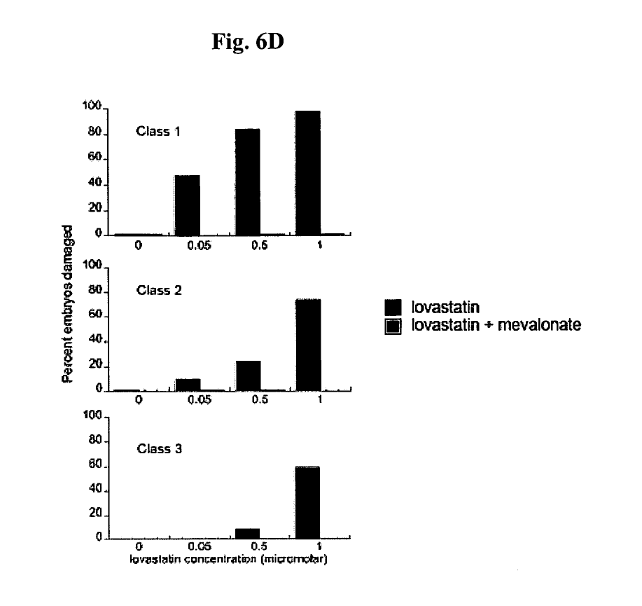

As in mammalian cell culture, lovastatin leads to clear dose-dependent muscle phenotypes in a zebrafish in vivo model of statin-induced muscle damage, demonstrated by longitudinal muscle fiber staining with an antibody to myosin heavy chain (FIGS. 6A, 6B, and 6D; Hanai et al., J. Clin. Invest. 117:3940-3951, 2007).

Muscle damage at low lovastatin concentration (0.025-0.05 μM) is evidenced by bowing, gap formation, and fiber disruption (class 1 changes). At higher lovastatin concentrations (0.05-0.5 μM), fiber damage is more severe. Fiber thinning and attenuation of staining with the MHC antibody is frequently seen (class 2 changes). At maximal lovastatin concentration (1.0-5.0 μM), damage beyond the muscle is observed, with the development of irregular somite boundries (class 3 changes). Zebrafish also bear an atrogin-1 gene 75% homologous at the amino acid level to the human counterpart, and previous studies have shown that knockdown of z-atrogin-1 prevents statin-induced muscle damage in the fish (Hanai et al., supra). As in mammalian myotube culture, 100 μM mevalonate completely prevented the development of zebrafish myofiber damage and zebrafish atrogin-1 induction following 12-hour treatment with 0.05 to 1.0 μM lovastatin (FIGS. 6A-6D). These data demonstrate that the effects of lovastatin on muscle morphology and atrogin-1 induction occur by pathways dependent on HMG CoA reductase function.

### Experimental Methods

In addition to methods described above, the following methods were used:

Zebrafish lines and maintenance: Adult zebrafish (Danio rerio) were maintained as described under standard laboratory conditions at 28.5° C. in a 14 hour light/10 hour dark cycle (Westerfield, in Institute of Neuroscience University of Oregon, Eugene, Oreg.). Developmental stages were determined by embryo morphology and hours post fertilization (hpf) (Kimmel et al., Dev. Dyn. 203:253-310, 1995). To examine the effects of lovastatin, the embryos at 20-24 hpf were immersed in the embryonic water (500 μM NaCl, 170 μM KCl, 330 μM CaCl₂, and 330 μM MgSO₄) at a concentration of 0.005-10 μM lovastatin (mevinolin) (Sigma) including 0.003% 1-phenyl-2-thiourea (Sigma) to inhibit pigmentation in a 24-well plate. After 32 hpf, the embryos were fixed by 4% paraformaldehyde in PBS.

Western blotting: Zebrafish embryos were homogenized in SDS sample buffer (30 embryos/30 μL of sample buffer) with microfuge pestle until the lysate became uniform in consistency and there was no longer stringy. The lysate was boiled for 5 minutes and centrifuged supernatant was processed for Western blotting (Hanai et al., J. Cell Biol. 158:529-539, 2002). Glyceraldehyde-3-phosphatase dehydrogenase (GAPDH) antibody was from Santa Cruz Biotechnology.

Whole zebrafish antibody staining: Zebrafish embryos were fixed by 4% paraformaldehyde in PBS overnight. After fixation, the embryos were washed by PBS and stored for at least 1 hour at −20° C. in methanol, and permeabilized for 30 minutes at −20° C. in acetone. Embryos were incubated with blocking buffer (1% BSA, 0.1% Tween-20 in PBS), and incubated with diluted primary antibody, anti-slow twitch myosin F59 (1:100; Developmental Studies Hybridoma Bank (DSHB), Department of Biological Sciences, University of Iowa) (Crow et al., Dev. Biol. 118:333-342, 1986; Devoto et al., Development 122:3371-3380, 1996) in blocking solution for overnight at 4° C. Staining was detected by using Alexa Fluor(R) 594 goat anti-mouse IgG (H+L) (1:200; Invitrogen) in blocking solution for 4 hours at room temperature (Birely et al., Dev. Biol. 280:162-176, 2005).

### Example 3

### Geranylgeranol Specifically Rescues Statin-Induced Muscle Damage

In addition to producing cholesterol, mevalonate is a building block of the polyprenyl tails conjugated to many intracellular proteins (e.g., farnesyl and geranylgeranyl protein modifications). Some proteins, such as lamins and ras, are derivatized by farnesyl phyrophosphate, a 15-carbon adduct generated from three mevalonate moieties. Other proteins, such as rho, rac, rab, and rapl, are derivatized by geranylgeranyl pyrophosphate, a 20-carbon adduct generated from four mevalonate moieties. Inhibition of squalene synthase and squalene epoxidase, distal enzymes to only cholesterol biosynthesis do not cause toxicity in cultured muscle cells (Flint et al., Toxicol. Appl. Pharmacol. 145:91-98, 1997; Matzno et al., J. Lipid Res. 38:1639-1648, 1997) and therefore, inhibition of cholesterol biosynthesis is not thought to mediate statin-induced muscle damage. Experiments were performed to determine whether statin inhibition of famesylation or geranylgeranylation may contribute to statin-induced atrogin-1 upregulation and muscle damage.

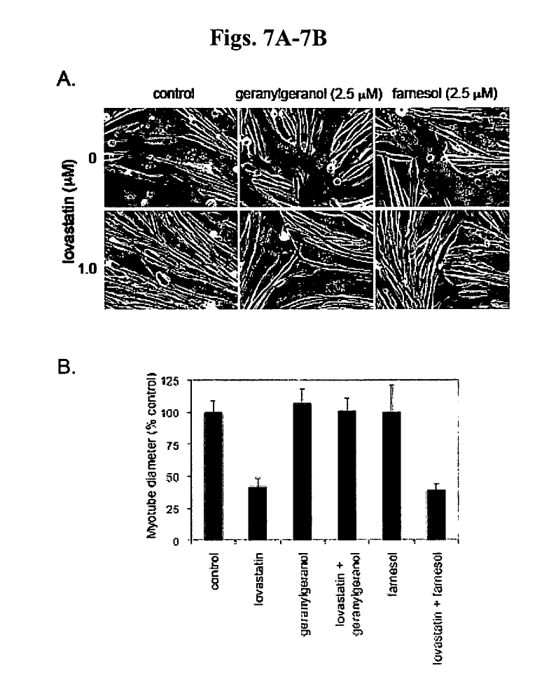

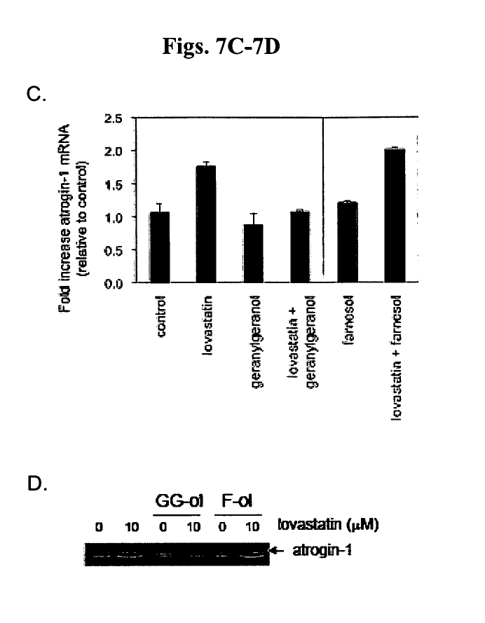

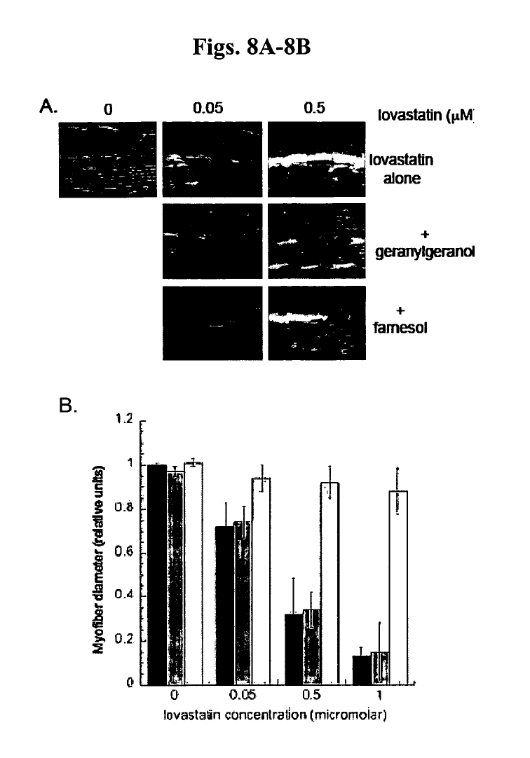

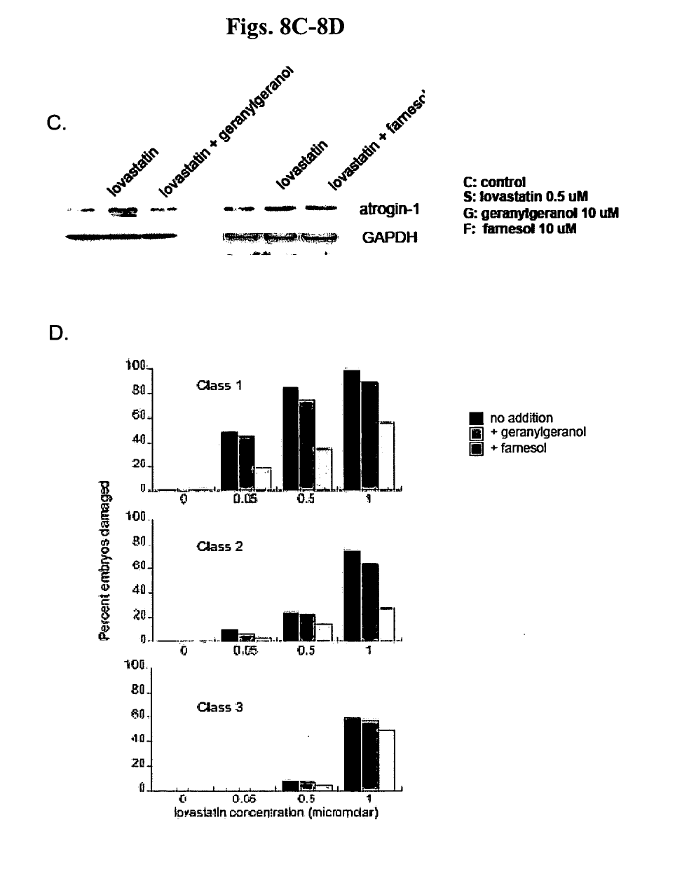

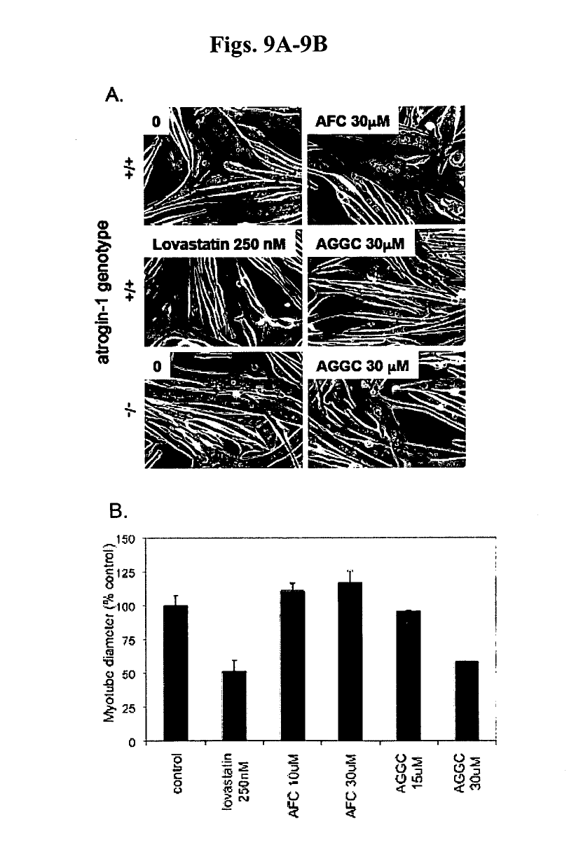

In a first set of experiments, farnesol or geranylgeranol, the cell-permeable precursors of famesyl pyrophosphate and geranylgeranyl pyrophosphate, respectivey (Crick et al., Biochem. Biophys. Res. Comm. 237:483-487, 1997) were added to statin-treated mammalian muscle culture or zebrafish embros to determine whether these precursors would rescue statin-induced muscle damage and block statin-induced atrogin-1 induction. In a series of control experiments, neither 2.5 μM farnesol nor 2.5 μM geranylgeranol alone had any effect on fiber diameter or atrogin-1 expression in myotube culture (FIGS. 7A-7D) or in zebrafish embryos (FIGS. 8A-8D) after 24-hour and 12-hour treatment, respectively. In contrast, when farnesol and geranylgeranol were administered in conjunction with lovastatin (24-hours for myotube culture and 12-hours for zebrafish embryos), 2.5 μM geranylgeranol, but not 2.5 μM farnesol reduced the degree of atrogin-1 mRNA and protein induction, as well as fiber damage in both models (FIGS. 7A-7D and FIGS. 8A-8D). The data demonstrate that proteins that require geranylgeranylation for their activity mediate statin toxicity in muscle, at least in part by causing atrogin-1 induction.

### Experimental Methods

The methods used to perform these experiments are described above. The additional reagents of farnesol and geranylgeranol were purchased from Sigma.

### Example 4

### A Geranylation Pathway Inhibitor Mimics Statin-Induced Muscle Damage

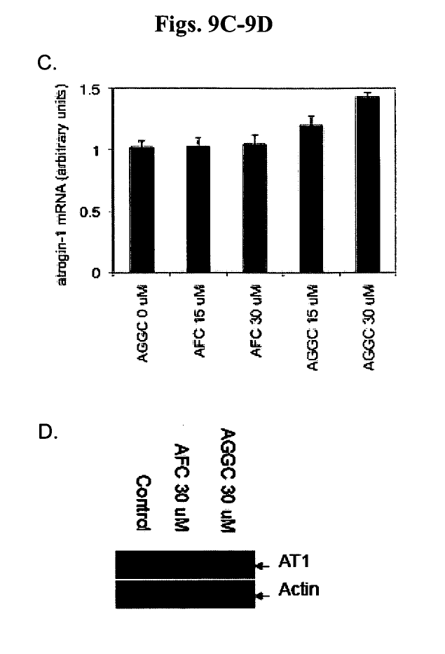

Additional evidence that inhibition of geranylgeranylation is critical to the toxicity of statins in muscle comes from experiments in which myotubes were treated with N-acetyl-S-geranylgeranyl-L-cysteine (AGGC), which inhibits processing of geranylgeranylated proteins by isoprenylcysteine carboxyl methyltransferase (ICMT). Treatment of mammalian myotubes with 30 μM AGGC for 24 hours caused a similar reduction of myotube diameter as 24-hour treatment with 0.25 μM lovastatin, and resulted in induction of both atrogin-1 mRNA and protein expression (FIGS. 9A-9D). This effect on myotubes was dependent on expression of atrogin-1, since myotubes derived from atrogin-1 knockout mice (−/−) were resistant to this reagent (FIG. 9A). In contrast, 30 μM N-acetyl-S-farnesyl-L-cysteine (AFC), which inhibits processing of farnesylated proteins by ICMT, had no effect on myotube integrity or atrogin-1 mRNA or protein levels (FIGS. 9A-9D).

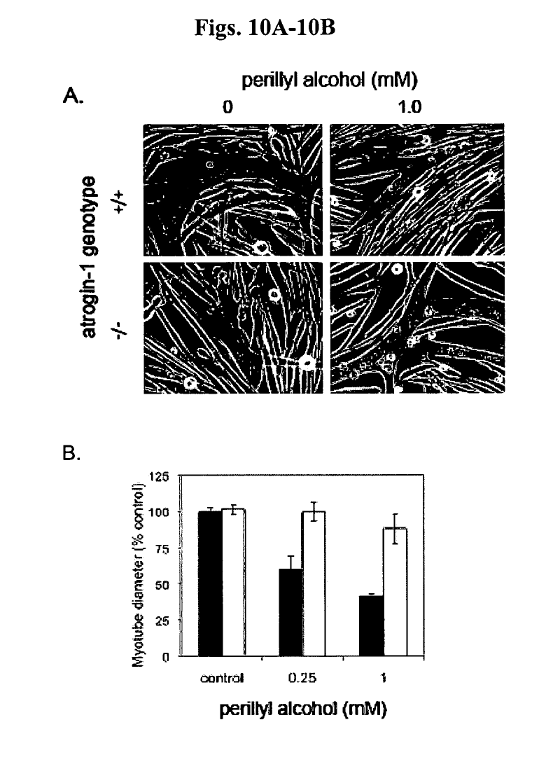

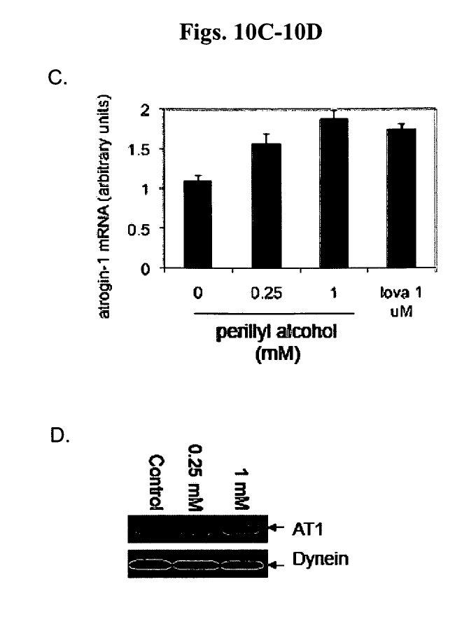

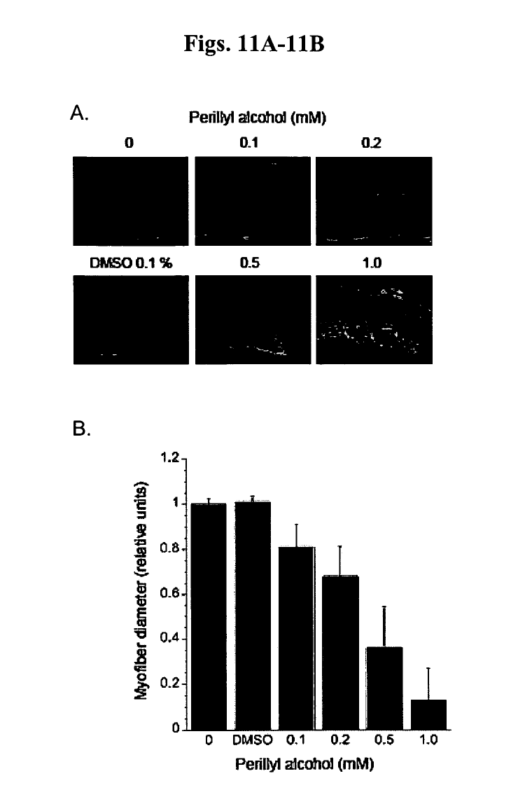

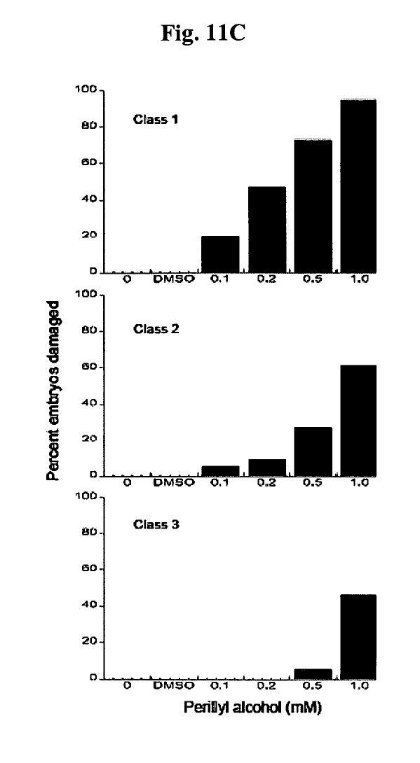

In a separate set of experiments, perillyl alcohol, an inhibitor of geranylgeranyl transferases (Ren et al., Biochem. Pharmacol. 54:113-120, 1997) was used to treat myotubes from wildtype or atrogin-1 knockout mice (−/−). Perillyl alcohol treatment (1.0 mM for 24-hours) led to marked damage of wildtype, but not atrogin-1 knockout myotubes, as well as induction of atrogin-1 mRNA and protein levels at similar concentrations as lovastatin in the wildtype myotubes (FIGS. 10A-10D). Perillyl alcohol had similar effects in zebrafish embryos. Perillyl alcohol treatment (0.1 to 1.0 mM perillyl alcohol for 12 hours) resulted in dramatic thinning of muscle fibers and morphologic damage in zebrafish embryos (FIGS. 11A-11C).

### Experimental Methods

The methods used to perform these experiments are described above. The additional reagents of AFC and AGGC were purchased from Sigma.

### Example 5

### Knockdown of Geranylgeranyl Transferase Mimics Statin-Induced Muscle Damage

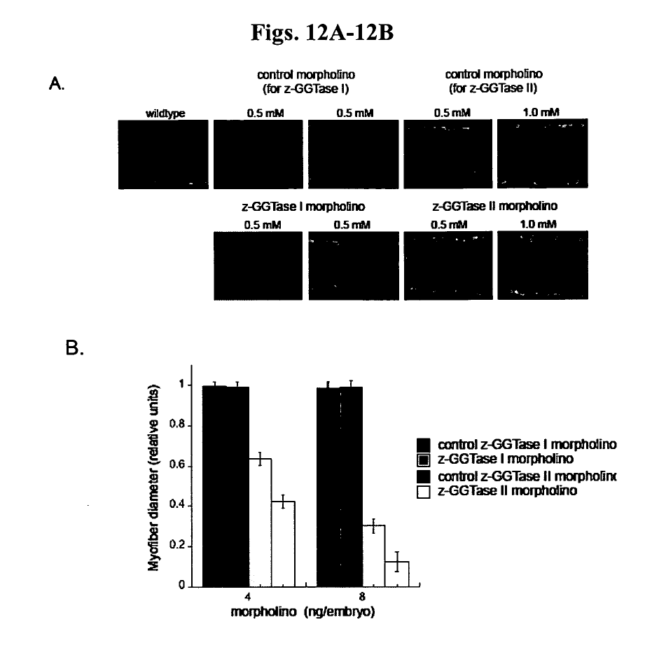

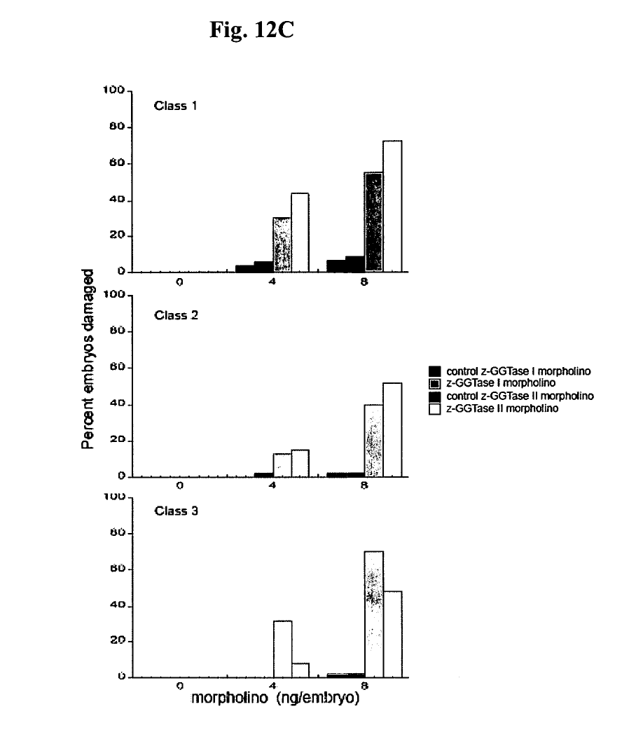

To confirm that the effect of perillyl alcohol on muscle fibers is due to inhibition of geranylgeranyl transferases and not a non-specific effect, experiments were performed to knockdown the beta subunit of geranylgeranyl transferase (GGTase) I and GGTase II in zebrafish embryos using antisense morpholino oligonucleotides targeting the ATG region of each gene (ATG morpholino). Depletion of both z-GGTases I and II demonstrated similar effects as perillyl alcohol and lovastatin treatment in zebrafish fibers (FIGS. 12A-12C). These data provide evidence that geranylgeranylated proteins mediate lovastatin-induced atrogin-1 induction and muscle damage, and that activation of protein geranylgeranylation and/or geranylgeranylation biosynthetic pathways will reduce or ameliorate statin-induced muscle damage.

### Experimental Methods

In addition to methods described above, the following methods were used:

Antisense morpholino oligonucleotide sequences and injection: Morpolino antisense oligonucleotides (MOs) were designed and synthesized by Gene Tools LLC. The MO used for knockdown of the z-GGTase I, beta subunit was 5′-AAT CCA CCG ACT CAA AAT CCG CCA T-3′ (SEQ ID NO: 6), and the five-base mismatch control for the z-GGTase I MO was 5′-AAT CCA GCC AGT CAA AAT GCC CCA T-3′ (SEQ ID NO: 7). The MO used for knockdown of the z-GGTase II, beta subunit was 5′-CTG ACT TCA GCC GTC ACA CAT ATA T-3′ (SEQ ID NO: 8), and the five-base mismatch control for the z-GGTase II MO was 5′-CTC ACT TGA GCG GTC AGA CAT AAA T-3′ (SEQ ID NO: 9). For each injection, MOs were diluted to 500 or 1000 μM. MOs were injected at 8 to 16 ng per embryo into one cell-stage embryos at the yolk and cytoplasm interface.

### OTHER EMBODIMENTS

All publications, patents, and patent applications cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art. Other embodiments are within the claims.

What is claimed is:

1. A method of treating or reducing the likelihood of developing a statin-mediated myopathy in a subject, said method comprising administering to said subject a therapeutically effective amount of a geranylgeranylation activator in an amount and for a time sufficient to treat or reduce the likelihood of developing said statin-mediated myopathy in said subject.

2. The method of claim 1, wherein said activator is mevalonate, phosphomevalonate, 5-pyrophosphomevalonate, isopentenyl pyrophosphate, dimethylallyl pyrophosphate, farnesyl diphosphate, geranyl diphosphate, geranylgeranyl pyrophosphate, or geranylgeranol.

3. The method of claim 2, wherein said activator is mevalonate or geranylgeranol.

4. The method of claim 1, wherein said administering results in at least a 10% reduction in one or more symptoms of a statin-mediated myopathy in the subject or in the likelihood of developing one or more symptoms of a statin-induced myopathy in the subject.

5-6. (canceled)

7. The method of claim 4, wherein said one or more symptoms of a statin-induced myopathy is selected from the group consisting of increased atrogin-1 expression or biological activity, increased creatine kinase (CK) enzyme levels, overt necrosis of myocytes, myalgia, myositis, myoskeletal pain, muscle pain, or increased microglobinuria or transaminase levels.

8. The method of claim 1, wherein said subject has increased atrogin-1 expression compared to a subject that does not have a myopathy or is not receiving a statin.

9. (canceled)

10. The method of claim 1, wherein said activator is administered orally or parenterally.

11-13. (canceled)

14. The method of claim 1, wherein said activator is administered in a dose of between 10 μg/day to 500 mg/day.

15. (canceled)

16. The method of claim 1, wherein said subject is administered a statin.

17. (canceled)

18. The method of claim 16, wherein said activator is administered following administration of said statin, or following cessation or termination of treatment with said statin.

19. The method of claim 16, wherein said activator is administered simultaneously or sequentially with a statin.

20. The method of claim 16, wherein said statin is selected from the group consisting of simvastatin, atrovastatin, fluvastatin, pravastatin, rosuvastatin, pitavastatin, lovastatin, compactin, mevinolin, mevastatin, velostatin, synvinolin, rivastatin, and cerivastatin.

21-28. (canceled)

29. A kit comprising: i) a geranylgeranylation activator; and ii) instructions for administration of said activator for the treatment of a statin-induced myopathy.

30. The kit of claim 29, wherein said activator is mevalonate, phosphomevalonate, 5-pyrophosphomevalonate, isopentenyl pyrophosphate, dimethylallyl pyrophosphate, farnesyl diphosphate, geranyl diphosphate, geranylgeranyl pyrophosphate, or geranylgeranol.

31-33. (canceled)

34. The kit of claim 29, wherein said activator is formulated for oral or parenteral administration.

35-39. (canceled)

40. The kit of claim 29, further comprising a statin and instructions for administration of said statin for the treatment of a statin-induced myopathy.

41-50. (canceled)

51. A kit comprising: i) a composition comprising one or more geranylgeranylation activators and one or more statin compounds; and ii) instructions for administration of said composition for the treatment of a statin-induced myopathy.

52. The kit of claim 51, wherein said composition is formulated as a bilayer tablet.

53. A composition comprising one or more geranylgeranylation activators and one or more statin compound.

54. The composition of claim 53, wherein said composition is formulated as a bilayer tablet.
